SEQUENCE LISTING (61 sequences)

SEQ ID NO: 1 - protein, 832 aa, synthetic construct [sequence omitted]

SEQ ID NO: 2 - protein, 832 aa, synthetic construct
  REGION 1..832: note = Taq E507K
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 3 - protein, 832 aa, synthetic construct
  REGION 1..832: note = Taq R536K
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYKELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 4 - protein, 832 aa, synthetic construct
  REGION 1..832: note = Taq R660V
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRVAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 5 - protein, 832 aa, synthetic construct
  REGION 1..832: note = Taq R536K/R660V
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYKELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRVAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - protein, 832 aa, synthetic construct
  REGION 1..832: note = Taq E507K/R536K
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYKELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - protein, 832 aa, synthetic construct
  REGION 1..832: note = Taq E507K/R660V
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRVAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 8 - protein, 832 aa, synthetic construct
  REGION 1..832: note = Taq E507K/R536K/R660V
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKEKKTGKRSTSAAVLEALREAHPIVEKILQYKELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRVAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 9 - DNA, 2499 bp, synthetic construct
  misc_feature 1..2499: note = Thermus aquaticus DNA polymerase (Taq)
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 10 - DNA, 2499 bp, synthetic construct
  misc_feature 1..2499: note = Taq E507K
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 11 - DNA, 2499 bp, synthetic construct
  misc_feature 1..2499: note = Taq R536K
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtacaaggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 12 - DNA, 2499 bp, synthetic construct
  misc_feature 1..2499: note = Taq R660V
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgcgtggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 13 - DNA, 2499 bp, synthetic construct
  misc_feature 1..2499: note = Taq R536K/R660V
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtacaaggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgcgtggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 14 - DNA, 2499 bp, synthetic construct
  misc_feature 1..2499: note = Taq E507K/R536
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtacaaggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 15 - DNA, 2499 bp, synthetic construct
  misc_feature 1..2499: note = Taq E507K/R660V
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgcgtggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 16 - DNA, 2499 bp, synthetic construct
  misc_feature 1..2499: note = Taq E507K/R536K/R660V
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtacaaggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgcgtggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 17 - DNA, 20 bp, synthetic construct
  misc_feature 1..20: note = Eco-forward primer
  source 1..20: mol_type = other DNA
ggggtacctcatcaccccgg

SEQ ID NO: 18 - DNA, 27 bp, synthetic construct
  misc_feature 1..27: note = R536K-reverse primer
  source 1..27: mol_type = other DNA
cttggtgagctccttgtactgcaggat

SEQ ID NO: 19 - DNA, 27 bp, synthetic construct
  misc_feature 1..27: note = R536K-forward primer
  source 1..27: mol_type = other DNA
atcctgcagtacaaggagctcaccaag

SEQ ID NO: 20 - DNA, 30 bp, synthetic construct
  misc_feature 1..30: note = R660V-reverse primer
  source 1..30: mol_type = other DNA
gatggtcttggccgccacgcgcatcagggg

SEQ ID NO: 21 - DNA, 30 bp, synthetic construct
  misc_feature 1..30: note = R660V-forward primer
  source 1..30: mol_type = other DNA
cccctgatgcgcgtggcggccaagaccatc

SEQ ID NO: 22 - DNA, 32 bp, synthetic construct
  misc_feature 1..32: note = Xba-reverse primer
  source 1..32: mol_type = other DNA
gctctagactatcactccttggcggagagcca

SEQ ID NO: 23 - DNA, 27 bp, synthetic construct
  misc_feature 1..27: note = E507K-reverse primer
  source 1..27: mol_type = other DNA
cttgccggtctttttcgtcttgccgat

SEQ ID NO: 24 - DNA, 27 bp, synthetic construct
  misc_feature 1..27: note = E507K-forward primer
  source 1..27: mol_type = other DNA
atcggcaagacgaaaaagaccggcaag

SEQ ID NO: 25 - DNA, 25 bp, synthetic construct
  misc_feature 1..25: note = rs1408799 forward primer
  source 1..25: mol_type = other DNA
ccagtgttaggttatttctaacttg

SEQ ID NO: 26 - DNA, 19 bp, synthetic construct
  misc_feature 1..19: note = rs1408799 reverse_T primer
  source 1..19: mol_type = other DNA
gctcggagcacatggtcaa

SEQ ID NO: 27 - DNA, 19 bp, synthetic construct
  misc_feature 1..19: note = rs1408799 reverse_C primer
  source 1..19: mol_type = other DNA
gctcggagcacatggtcag

SEQ ID NO: 28 - DNA, 22 bp, synthetic construct
  misc_feature 1..22: note = rs1015362 forward primer
  source 1..22: mol_type = other DNA
tgaagagcaggaaagttcttca

SEQ ID NO: 29 - DNA, 20 bp, synthetic construct
  misc_feature 1..20: note = rs1015362 reverse_C primer
  source 1..20: mol_type = other DNA
actgtgtgtctgaaacagtg

SEQ ID NO: 30 - DNA, 20 bp, synthetic construct
  misc_feature 1..20: note = rs1015362 reverse_T primer
  source 1..20: mol_type = other DNA
actgtgtgtctgaaacagta

SEQ ID NO: 31 - DNA, 26 bp, synthetic construct
  misc_feature 1..26: note = rs4911414 forward_G primer
  source 1..26: mol_type = other DNA
gtaagtctttgctgagaaattcattg

SEQ ID NO: 32 - DNA, 26 bp, synthetic construct
  misc_feature 1..26: note = rs4911414 forward_T primer
  source 1..26: mol_type = other DNA
gtaagtctttgctgagaaattcattt

SEQ ID NO: 33 - DNA, 23 bp, synthetic construct
  misc_feature 1..23: note = rs4911414 reverse primer
  source 1..23: mol_type = other DNA
agtatccagggttaatgtgaaag

SEQ ID NO: 34 - DNA, 25 bp, synthetic construct
  misc_feature 1..25: note = 1408799-FAM probe
  source 1..25: mol_type = other DNA
agatatttgtaaggtattctggcct

SEQ ID NO: 35 - DNA, 24 bp, synthetic construct
  misc_feature 1..24: note = 1015362-HEX probe
  source 1..24: mol_type = other DNA
tgctgaacaaatagtcccgaccag

SEQ ID NO: 36 - DNA, 24 bp, synthetic construct
  misc_feature 1..24: note = 4911414-Texas Red probe
  source 1..24: mol_type = other DNA
tttctctagttgcctttaagattt

SEQ ID NO: 37 - protein, 832 aa, synthetic construct
  REGION 1..832: note = Taq E507K/R536K/R587I/R660V
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKEKKTGKRSTSAAVLEALREAHPIVEKILQYKELTKLKSTYIDPLPDLIHPRTGRLHTRFNQTATATGRLSSSDPNLQNIPVITPLGQRIRRAFIAEEGWLLVALDYSQIELRVLAHLSGDENLIRVFQEGRDIHTETASWMFGVPREAVDPLMRVAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 38 - DNA, 2499 bp, synthetic construct
  misc_feature 1..2499: note = Taq E507K/R536K/R587I/R660V
  source 1..2499: mol_type = other DNA
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacgaaaaagaccggcaagcgctccaccagcgccgccgtcctggaggccctccgcgaggcccaccccatcgtggagaagatcctgcagtacaaggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtcatcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgcgtggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 39 - DNA, 36 bp, synthetic construct
  misc_feature 1..36: note = Kpn-forward primer
  source 1..36: mol_type = other DNA
tccaccccgaggggtacctcatcaccccggcctggc

SEQ ID NO: 40 - DNA, 27 bp, synthetic construct
  misc_feature 1..27: note = R587I-reverse primer
  source 1..27: mol_type = other DNA
cccaagcggggtgatgacggggatgtt

SEQ ID NO: 41 - DNA, 27 bp, synthetic construct
  misc_feature 1..27: note = R587I-forward primer
  source 1..27: mol_type = other DNA
aacatccccgtcatcaccccgcttggg

SEQ ID NO: 42 - DNA, 37 bp, synthetic construct
  misc_feature 1..37: note = Xba-reverse primer
  source 1..37: mol_type = other DNA
ctgcaggtcgactctagactatcactccttggcggag

SEQ ID NO: 43 - DNA, 24 bp, synthetic construct
  misc_feature 1..24: note = KRAS Q61H forward_Q primer(24mer)
  source 1..24: mol_type = other DNA
gatattctcgacacagcaggtcaa

SEQ ID NO: 44 - DNA, 24 bp, synthetic construct
  misc_feature 1..24: note = KRAS Q61H forward_H primer(24mer)
  source 1..24: mol_type = other DNA
gatattctcgacacagcaggtcac

SEQ ID NO: 45 - DNA, 20 bp, synthetic construct
  misc_feature 1..20: note = KRAS Q61H reverse primer
  source 1..20: mol_type = other DNA
acaaagaaagccctccccag

SEQ ID NO: 46 - DNA, 25 bp, synthetic construct
  misc_feature 1..25: note = KRAS Q61H FAM probe
  source 1..25: mol_type = other DNA
tgcaatgagggaccagtacatgagg

SEQ ID NO: 47 - DNA, 18 bp, synthetic construct
  misc_feature 1..18: note = KRAS Q61H forward_Q primer(18mer)
  source 1..18: mol_type = other DNA
ctcgacacagcaggtcaa

SEQ ID NO: 48 - DNA, 18 bp, synthetic construct
  misc_feature 1..18: note = KRAS Q61H forward_H primer(18mer)
  source 1..18: mol_type = other DNA
ctcgacacagcaggtcac

SEQ ID NO: 49 - DNA, 20 bp, synthetic construct
  misc_feature 1..20: note = KRAS Q61H reverse primer
  source 1..20: mol_type = other DNA
acaaagaaagccctccccag

SEQ ID NO: 50 - DNA, 21 bp, synthetic construct
  misc_feature 1..21: note = KRAS G13D forward primer
  source 1..21: mol_type = other DNA
ataaggcctgctgaaaatgac

SEQ ID NO: 51 - DNA, 17 bp, synthetic construct
  misc_feature 1..17: note = KRAS G13D reverse_G primer(17mer)
  source 1..17: mol_type = other DNA
ggcactcttgcctacgc

SEQ ID NO: 52 - DNA, 17 bp, synthetic construct
  misc_feature 1..17: note = KRAS G13D reverse_D primer(17mer)
  source 1..17: mol_type = other DNA
ggcactcttgcctacgt

SEQ ID NO: 53 - DNA, 30 bp, synthetic construct
  misc_feature 1..30: note = G1213_R FAM
  source 1..30: mol_type = other DNA
agctccaactaccacaagtttatattcagt

SEQ ID NO: 54 - DNA, 23 bp, synthetic construct
  misc_feature 1..23: note = KRAS G12S forward_G primer(23mer)
  source 1..23: mol_type = other DNA
taaacttgtggtagttggagctg

SEQ ID NO: 55 - DNA, 23 bp, synthetic construct
  misc_feature 1..23: note = KRAS G12S forward_S primer(23mer)
  source 1..23: mol_type = other DNA
taaacttgtggtagttggagcta

SEQ ID NO: 56 - DNA, 28 bp, synthetic construct
  misc_feature 1..28: note = KRAS G12S reverse primer
  source 1..28: mol_type = other DNA
catattcgtccacaaaatgattctgaat

SEQ ID NO: 57 - DNA, 25 bp, synthetic construct
  misc_feature 1..25: note = G1213_F FAM probe
  source 1..25: mol_type = other DNA
agctgtatcgtcaaggcactcttgc

SEQ ID NO: 58 - DNA, 20 bp, synthetic construct
  misc_feature 1..20: note = EGFR L858R forward primer
  source 1..20: mol_type = other DNA
acctggcagccaggaacgta

SEQ ID NO: 59 - DNA, 19 bp, synthetic construct
  misc_feature 1..19: note = EGFR L858R reverse_L primer
  source 1..19: mol_type = other DNA
gcacccagcagtttggcca

SEQ ID NO: 60 - DNA, 19 bp, synthetic construct
  misc_feature 1..19: note = EGFR L858R reverse_R primer
  source 1..19: mol_type = other DNA
gcacccagcagtttggccc

SEQ ID NO: 61 - DNA, 28 bp, synthetic construct
  misc_feature 1..28: note = L858R FAM_R probe
  source 1..28: mol_type = other DNA
cagcatgtcaagatcacagattttgggc

(19) United States
(12) Patent Application Publication
(10) Pub. No.: US 2024/0076633 A1
(43) Pub. Date: Mar. 7, 2024

(54) PCR BUFFER COMPOSITION FOR INCREASING ACTIVITY OF DNA POLYMERASE WITH INCREASED GENE MUTATION SPECIFICITY

(71) Applicant: GENECAST CO., LTD., Seongnam-si (KR)

(72) Inventors: Byung Chul LEE, Hanam-si (KR); Il Hyun PARK, Hanam-si (KR); Huy Ho LEE, Seongnam-si (KR)

(21) Appl. No.: 18/478,874

(22) Filed: Sep. 29, 2023

Related U.S. Application Data
(63) Continuation of application No. 16/630,229, filed on Jan. 10, 2020.

(30) Foreign Application Priority Data
Jul. 12, 2017 (KR) 10-2017-0088373
Jul. 12, 2017 (KR) 10-2017-0088376

(51) Int. Cl. C12N 9/12; C12N 15/70; C12Q 1/6827; C12Q 1/686; C12Q 1/6883

(52) U.S. Cl. CPC C12N 9/1252; C12N 15/70; C12Q 1/6827; C12Q 1/686; C12Q 1/6883; C12Y 207/07007; C12Q 2600/156

(57) ABSTRACT

Provided are a PCR buffer composition for increasing the activity of a DNA polymerase having increased gene mutation specificity, a PCR kit for detecting a gene mutation or SNP comprising the PCR buffer composition and/or the DNA polymerase having increased gene mutation specificity, and a method for in vitro detecting one or more gene mutations or SNPs in one or more templates by using the kit.

### TECHNICAL FIELD

The present invention relates to a DNA polymerase with increased gene variation specificity and a PCR buffer composition for increasing the activity thereof, and more specificity, a DNA polymerase with increased gene variation specificity due to a mutation occurring at a specific amino acid position, a nucleic acid sequence encoding the polymerase, a vector including the nucleic acid sequence and a host cell transformed with the vector, a method of in vitro detecting one or more gene variations or SNPs in one or more templates using the DNA polymerase with increased gene variation specificity, a composition for detecting a gene variation or SNP, which includes the DNA polymerase, and a polymerase chain reaction (PCR) kit including the composition.

Moreover, the present invention provides a PCR buffer composition for increasing the activity of the DNA polymerase with increased gene variation specificity, a PCR kit for detecting a gene variation or SNP, which includes the PCR buffer composition and/or the DNA polymerase with increased gene variation specificity, and a method of in vitro detecting one or more gene variations or SNPs in one or more templates using the kit.

### REFERENCE TO SEQUENCE LISTING

The present application is being filed along with a Sequence Listing in electronic format. The Sequence Listing is provided as a file entitled 58338589_1.xml, created and last modified on Sep. 29, 2023, which is 105 Kb in size. The information in the electronic format of the Sequence Listing is incorporated herein by reference in its entirety.

### BACKGROUND ART

Since the first human genomic sequence has been defined, the inventors have focused on finding the genetic difference among individuals, such as single nucleotide polymorphisms (SNPs). SNPs in a genome are of interest because itis more and more dear that they are associated with different drug resistances or predisposing factors for various diseases. Due to the subsequent knowledge of medically-related nucleotide variation, a therapeutic method for the genetic supply of an individual may be applied, and a drug therapy which is ineffective or causes a side effect may be prevented. The development of technology that enables time- and cost-effective identification of nucleotide variation will bring further advances in pharmacogenetics.

SNPs account for the major genetic variations in a human genome and cause 90% or more of differences between individuals. To detect other nucleic acid variations such as the genetic variations and mutations, various methods may be used. For example, the identification of a variant of a target nucleic acid may be accomplished by hybridizing a nucleic acid sample to be analyzed with a hybridization primer specific for a sequence variant under suitable hybridization conditions

However, it was found that such a hybridization method cannot satisfy clinical needs, particularly, in terms of sensitivity, which is required for an assay. Therefore, PCR has been extensively used in molecular biology and a diagnostic testing method for detecting mutations such as SNPs and other allelic sequence variants. Here, in consideration of the presence of a variant, a target nucleic acid to be tested was amplified by polymerase chain reaction (PCR) before hybridization. As a hybridization probe for the assay, generally, a single-stranded oligonucleotide is generally used. A modified embodiment of the assay includes a fluorescent hybridization probe. Generally, efforts have been made to automate methods of measuring SNPs and other sequence variations (Gut, Hum. Mutat. 17, 475-492 (2001)).

An alternative to sequence variation-specific hybridization known in the art is provided by so-called gene variation-specific amplification. In this detection method, during amplification, a variation-specific amplification primer is used, and generally has a so-called differential terminal nucleotide residue at the 3′ end of the primer, where the residue is only complementary for one specific variation of a target nucleic acid to be detected. In this method, the nucleotide variant is measured by the presence or absence of a DNA product after PCR amplification. The principle of gene variation-specific amplification is based on the formation of a canonical or non-canonical primer-template complex at the end of a gene variation-specific amplification primer.

Precisely, at the 3′ end of the paired primer, amplification occurs by a DNA polymerase, but at the mismatched primer end, extension is suppressed.

For example, U.S. Pat. No. 5,595,890 discloses a method for gene variation-specific amplification and its application thereof, for example, the application to detect clinically associated point mutation in a k-ras tumor gene. In addition, U.S. Pat. No. 5,521,301 discloses an allele-specific amplification method for genotyping of an ABO blood group system. In contrast, U.S. Pat. No. 5,639,611 discloses the use of allele-specific amplification associated with the detection of a point mutation that causes sickle cell anemia. However, gene variation-specific amplification or allele-specific amplification is problematic in that it has low selectivity, and thus a more complicated and time-and cost-intensive optimizing step is needed.

Such a method for detecting sequence variations, polymorphisms and mainly point mutations requires allele--specific amplification (or gene variation-specific amplification) particularly when a sequence variation to be detected is deficient compared to dominant variations in the same nucleic acid fragment (or the same gene).

For example, this situation occurs when sporadic tumor cells are detected in the body fluid such as blood, serum or plasma by gene variation-specific amplification (U.S. Pat. No. 5,496,699). To this end, DNA is first isolated from the body fluid such as blood, serum or plasma, and DNA is derived from deficient, sporadic tumor cells and excessive non-proliferative cells. Thus, mutations that are significant to tumor DNA in the k-ras gene should be detected from several copies in the presence of an excessive amount of wild-type DNA.

All methods for gene variation-specific amplification disclosed in the prior art have the disadvantage that a 3′-terminal differential oligonucleotide residue should be used. In addition, despite the use of a 3′-differential nucleotide residue, these methods have the disadvantage that primer extension occurs at low levels in the presence of a suitable DNA polymerase even when a target nucleic acid is not exactly matched with a sequence variant to be detected. Particularly, when a specific sequence variant is detected by an excessive background nucleic acid including a different sequence variant, it leads to a false positive result. The main reason for the disadvantage of the PCR-based method is the incompatibility of a polymerase used in the method for sufficiently differentiating mismatched bases. Therefore, it is not yet possible to directly obtain clear data on the presence or absence of a mutation by PCR. To date, additional time- and cost-intensive purification and analysis methods have been required for the clear diagnosis of mutations. Therefore, a novel method for improving the selectivity of gene variation- or allele-specific PCR amplification will greatly affect the reliability and robustness of direct gene variation or SNP analysis by PCR.

Therefore, there are continuous demands for the development of a DNA polymerase with increased gene variation specificity and an optimal reaction buffer in which various materials are mixed to exhibit a proper function of the DNA polymerase.

The inventors had made efforts to develop a novel DNA polymerase that can improve the selectivity of gene variation-specific PCR amplification and a reaction buffer for increasing its activity, confirming that gene variation specificity significantly increased when a mutation occurs at an amino acid residue at a specific position of Taq polymerase, and the activity of the DNA polymerase with increased gene variation specificity increases when the concentration of KCl, (NH₄)₂SO₄ and/or tetra methyl ammonium chloride (TMAC) am the components of the PCR buffer composition is adjusted, and thus the present invention was completed.

### DISCLOSURE

### Technical Problem

The present invention is directed to providing a DNA polymerase for detecting one or more gene variations or SNPs in a target sequence having a gene variation or SNP.

The present invention is also directed to providing a nucleic acid sequence encoding the DNA polymerase according to the present invention, a vector including the nucleic acid sequence, and a host cell transformed with the vector.

The present invention is also directed to providing a method of preparing the DNA polymerase according to the present invention.

The present invention is also directed to providing a method of in vitro detecting one or more gene variations or SNPs in one or more templates using the DNA polymerase of the present invention.

The present invention is also directed to providing a composition for detecting a gene variation or SNP, which includes the DNA polymerase of the present invention.

The present invention is also directed to providing a kit for detecting the DNA polymerase of the present invention, which includes the composition for detecting a gene variation or SNP according to the present invention.

The present invention is also directed to providing a PCR buffer composition for increasing the activity of a DNA polymerase with increased gene variation specificity.

The present invention is also directed to providing a PCR kit for detecting a gene variation or SNP, which includes the PCR buffer composition and/or the DNA polymerase with increased gene variation specificity according to the present invention.

The present invention is also directed to providing a method of in vitro detecting one or snore gene variations or SNPs in one or more templates using the PCR kit according to the present invention.

### Technical Solution

One aspect of the present invention provides a DNA polymerase comprising a Taq polymerase amino acid sequence of SEQ ID NO: 1, the DNA polymerase including

(a) a substitution at amino acid residue 507 in the amino acid sequence of SEQ ID NO: 1; and

(b) (i) a substitution at amino acid residue 536 in the amino acid sequence of SEQ ID NO: 1,

(ii) a substitution at amino acid residue 660 in the amino acid sequence of SEQ ID NO: 1.

(iii) substitutions at amino acid residues 536 and 660 in the amino acid sequence of SEQ ID NO: 1, or

(iv) substitutions at amino acid residues 536, 587 and 660 in the amino acid sequence of SEQ ID NO: 1.

According to an exemplary embodiment of the present invention, the substitution at the amino acid residue 507 may be a substitution of glutamic acid (E) with lysine (K), the substitution at the amino acid residue 536 is a substitution of arginine (R) with lysine (K), the substitution at the amino acid residue 587 is a substitution of arginine (R) with isoleucine (I), and the substitution at the amino acid residue 660 is a substitution of arginine (R) with valine (V).

According to another exemplary embodiment of the present invention, the DNA polymerase may discriminate a matched primer from a mismatched primer, wherein the matched primer may be hybridized with the target sequence, and the mismatched primer may have a non--canonical nucleotide at the 3′ end thereof with respect to the hybridized target sequence.

According to still another exemplary embodiment of the present invention, the DNA polymerase may exhibit a Ct value lower than the amplification of the target sequence comprising the mismatched primer.

Another aspect of the present invention provides a nucleic acid sequence encoding the DNA polymerase according to the present invention, a vector including the nucleic acid sequence, and a host cell transformed with the vector.

Still another aspect of the present invention provides a method of preparing a DNA polymerase, which includes: culturing the host cells; and isolating a DNA polymerase from the cell culture and a supernatant thereof.

Yet another aspect of the present invention provides a method of in vitro detecting one or more gene variations or SNPs in one or more templates, the method including:

bringing the DNA polymerase; according to the present invention into contact with a) one or more templates;

b) one or more matched primers, one or more mismatched primers or both of one or more matched primers and one or more mismatched primers; and

c) a nucleoside triphosphate,

wherein the one or more matched primers and the one or more mismatched primers are hybridized with a target sequence, and the mismatched primer has a non-canonical nucleotide at base position 7 from the 3′ end thereof with respect to the hybridized target sequence.

According to an exemplary embodiment of the present invention, the method may include a melting point analysis using a double strand-specific dye.

According to another exemplary embodiment of the present invention, the method may be accomplished by real-time PCR, the analysis on agarose gel after standard PCR, gene variation-specific amplification or allele-specific amplification through real-time PCR, tetra-primer amplification-refractory mutation system PCR or isothermal amplification.

Yet another aspect of the present invention provides a composition for detecting a gene variation or SNP, comprising the DNA polymerase according to the present invention.

Yet another aspect of the present invention provides a PCR kit including the composition for detecting a gene variation or SNP.

According to one exemplary embodiment of the present invention, the PCR kit may be used in competitive allele-specific TaqMan PCR (cast PCR), droplet digital PCR or MassARRAY.

According to another exemplary embodiment of the present invention, the PCR kit may further include one or more matched primers, one or more mismatched primers or both of one or more matched primers and one or more mismatched primers, where the one or more matched primers and the one or more mismatched primers may be hybridized with a target sequence, and the mismatched primer may have a non-canonical nucleotide at base position 7 from the 3′ end thereof with respect to the hybridized target sequence.

According to still another exemplary embodiment of the present invention, the PCR kit may further include a nucleoside triphosphate.

According to yet another exemplary embodiment of the present invention, the PCR kit may further include

a) one or more buffers;

b) a quantification reagent binding to double-stranded DNA;

c) a polymerase blocking antibody;

d) one or more control values or control sequences; and

e) one or more templates.

Yet another aspect of the present invention provides a PCR buffer composition for increasing the activity of a DNA polymerase with increased gene variation specificity, which includes 25 to 100 mM KCl; and 1 to 15 mM (NH₄)₂SO₄, wherein the final pH is 8.0 to 9.0.

According to one exemplary embodiment of the present invention, the KCl concentration may be 60 to 90 mM.

According to another exemplary embodiment of the present invention, the (NH₄)₂SO₄ concentration may be 2 to 8 mM.

According to still another exemplary embodiment of the present invention, the KCl concentration may be 70 to 80 mM, and the (NH₄)₂SO₄ concentration may be 4 to 6 mM.

Yet another aspect of the present invention provides a PCR buffer composition for increasing the activity of a DNA polymerase with increased gene variation specificity, which includes 5 to 80 mM TMAC in the above-described PCR buffer composition.

According to one exemplary embodiment of the present invention, the KCl concentration may be 40 to 90 mM.

According to another exemplary embodiment of the present invention, the (NH₄)₂SO₄ concentration may be 1 to 7 mM.

According to still another exemplary embodiment of the present invention, the TMAC concentration may be 15 to 70 mM, the KCl concentration may be 50 to 80 mM, and the (NH₄)₂SO₄ concentration may be 1.5 to 6 mM.

According to yet another exemplary embodiment of the present in the PCR buffer composition may further include Tris.Cl and MgCl₂.

Yet another aspect of the present invention provides a PCR kit for detecting a gene variation or SNP, which includes the above-described PCR buffer composition.

According to one exemplary embodiment of the present invention, the PCR kit may include a DNA polymerase comprising a Taq polymerase amino acid sequence of SEQ ID NO: 1, where the DNA polymerase has the substitutions) of the following amino acids:

(a) a substitution at amino acid residue 507 in the amino acid sequence of SEQ ID NO: 1; and

(b) (i) a substitution at amino acid residue 536 in the amino acid sequence of SEQ ID NO: 1,

(ii) a substitution at amino acid residue 660 in the amino acid sequence of SEQ ID NO: 1.

(iii) substitutions at amino acid residues 536 and 660 in the amino acid sequence of SEQ ID NO: 1, or

(iv) substitutions at amino acid residues 536, 587 and 660 in the amino acid sequence of SEQ ID NO: 1.

According to another exemplary embodiment of the present invention, the substitution at the amino acid residue 507 may be a substitution of glutamic acid (E) with lysine (K), the substitution at the amino acid residue 536 may be a substitution of arginine (R) with lysine (K), the substitution at the amino acid residue 587 may be a substitution of arginine (R) with isoleucine (I), and the substitution at the amino acid residue 660 may be a substitution of arginine (R) with valine (V).

According to still another exemplary embodiment of the present invention, the PCR kit may further include a) a nucleoside triphosphate; b) a quantification reagent binding to double-stranded DNA; c) a polymerase blocking antibody; d) one or more control values or control sequences; and e) one or more templates.

Yet another aspect of the present invention provides a method of in vitro detecting one or more gene variations or SNPs in one or more templates using the PCR kit of the present invention.

### Advantageous Effects

Since the DNA polymerase with increased gene variation specificity according to the present invention has a higher mismatch-to-match extension selectivity than conventional Taq polymerase, reliable gene variation-specific amplification is possible without any substrate modification. The present invention also provides an optimal PCR buffer composition that allows the proper function of a DNA polymerase with increased gene variation specificity to be effectively exhibited, and reliable gene variation-specific amplification is possible by considerably increasing the activity of the DNA polymerase using the DNA polymerase with increased gene variation specificity. Moreover, a kit including a PCR buffer composition and/or the DNA polymerase with increased gene variation specificity according to the present invention can effectively detect a gene variation or SNP, and thus can be usefully applied to the medical diagnosis of a disease and recombinant DNA studies.

### DESCRIPTION OF DRAWINGS

FIG. 1 shows a process of preparing Taq DNA polymerases having R536K, R660V and R536K/R660V variations: (a) the schematic representation of fragment PCR and overlap PCR; (b) the result of electrophoresis for amplified. products obtained by the fragment PCR; and (c) the result of electrophoresis for amplified products obtained by full-length amplification through overlap PCR.

FIG. 2 shows the result of electrophoresis for a pUC19 vector which is digested with restriction enzymes EcoRI/XbaI and treated with SAP and the purified overlap PCR products of (c) in FIG. 1.

FIG. 3 is the schematic representation of fragment PCR and overlap PCR during the preparation of Taq DNA polymerases having E507K, E507K/R536K, E507K/R660V and E507K/R536K/R660V variations, respectively.

FIG. 4 shows the result of electrophoresis for a pUC19 vector digested with EcoRI/XbaI and then treated with SAP and the purified overlap PCR product of FIG. 3 for gel extraction.

FIG. 5 is the schematic representation of the process of preparing a PCR template by collecting oral epithelial cells.

FIGS. 6a to 6d show the results of AS-qPCR for rs1408799 using Taq polymerases having E507K/R536K, E507K/R660V and E507K/R536K/R660V variations according to the present invention, and Taq polymerase having an E507K variation is used as a control.

FIGS. 7a to 7d show the results of AS-qPCR for rs1015362 using Taq polymerases having E507K/R536K, E507K/R660V and E507K/R536K/R660V variations according to the present invention, and Taq polymerase having an E507K variation is used as a control.

FIGS. 8a to 8d shows the results of AS-qPCR for rs1015362 using Taq polymerases having E507K/R536K, E507K/R660V and E507K/R536K/R660V variations according to the present invention, and Taq polymerase having an E507K variation is used as a control.

FIG. 9 shows the process of preparing Taq DNA polymerase having E507K/R536K/R587I/R660V variations: (a) the schematic representation of fragment PCR and overlap PCR; and (b) the result of electrophoresis for the amplified product obtained by the fragment PCR.

FIGS. 10a to 10d show the results of AS-qPCR for a template having an SNP of Q61H in a KRAS gene using a E507K/R536K/R587I/R660V polymerase, wherein FIGS. 10a and 10b show the results obtained using a 24-mer long primer, FIGS. 10c and 10d show the results obtained using a 18-mer long primer, and Taq polymerase having E507K/R536K/R660V variations is used as a control.

FIG. 11 shows the result obtained by AS-qPCR for a template having an SNP of G13D in a KRAS gene using a E507K/R536K/R587I/R660V polymerase, and Taq polymerase having E507K/R536K/R660V variations is used as a control.

FIG. 12 shows the result obtained by AS-qPCR for a template having an SNP of G12S in a KRAS gene using a E507K/R536K/R587I/R660V polymerase, and Taq polymerase having E507K/R536K/R660V variations is used as a control.

FIG. 13 shows the result obtained by AS-qPCR for a template having an SNP of L585R in an EGFR gene using a E507K/R536K/R587I/R660V polymerase, and Taq polymerase having E507K/R536K/R660V variations is used as a control.

FIGS. 14a to 14d are graphs showing the amplification delay effect by mismatch according to the change in KCl concentration of a reaction buffer using E507K, E507K/R536K, E507K/R660V and E507K/R536K/R660V Taq polymerases of the present invention.

FIG. 15 shows the result of electrophoresis for a PCR product obtained by amplification with a constantly fixed (NH₄)₂SO₄ concentration and various KCl concentrations to confirm the optimal KCl concentration in a reaction buffer.

FIG. 16 shows the result of electrophoresis for a PCR product obtained by amplification with a constantly fixed KCl concentration and various (NR₄)₂SO₄ concentrations to confirm the optimal (NH₄)₂SO₄ concentration in a reaction buffer.

FIG. 17 is the graph showing the amplification delay effect by mismatch according to the change in (NH₄)₂SO₄ concentration in a reaction buffer.

FIGS. 18a and 18b are graphs showing the amplification delay effect by mismatch according to the change in TMAC concentration after KCl and (NH₄)₂SO₄ concentrations are constantly fixed in a reaction buffer.

FIGS. 19a and 19b are graphs showing the amplification delay effect by mismatch according to the change in KCl concentration after TMAC and (NH₄)₂SO₄ concentrations are constantly fixed in a reaction buffer.

### NODES OF THE INVENTION

Hereinafter, the present invention will be described in further detail.

As described above, to improve the disadvantages of the gene variation-specific amplification method disclosed in the conventional art, there is a continuous demand for the development of a DNA polymerase with increased gene variation specificity and an optimal reaction buffer in which various materials are mixed such that the DNA polymerase can exhibit the proper function, and the development of such a method greatly affects the reliability and robustness of direct gene variation or SNP analysis by PCR. The inventors had made an effort to develop a novel DNA polymerase capable of improving the selectivity of gene variation-specific PCR amplification and a reaction buffer for increasing its activity, confirming that gene variation specificity significantly increased when a mutation occurs on an amino acid residue at a specific position of Taq polymerase, and the activity of the DNA polymerase with increased gene variation-specific amplification efficiency increases when the concentration of KCl, (NH₄)₂SO₄ and/or tetra methyl ammonium chloride (TMAC) among the components of the PCR buffer composition is adjusted, and thus the present invention was completed.

Since the DNA polymerase with increased gene variation specificity according to the present invention has a higher mismatch-to-match extension selectivity than conventional Taq polymerase, reliable gene variation-specific amplification is possible without any substrate modification. The present invention also provides an optimal PCR buffer composition that allows the proper function of a DNA polymerase with increased gene variation specificity to be effectively exhibited, and reliable gene variation-specific amplification is possible by considerably increasing the activity of the DNA polymerase using the DNA polymerase with increased gene variation specificity. Moreover, a kit including a PCR buffer composition and/or the DNA polymerase with increased gene variation specificity according to the present invention can effectively detect a gene variation or SNP, and thus can be usefully applied to the medical diagnosis of a disease and recombinant DNA studies.

Hereinafter, terms used herein will be defined.

The “amino acid” refers to any monomer unit that can be incorporated into a peptide, a polypeptide or a protein. The term “amino acid” used herein includes 20 natural or genetically encoded alpha-amino acids as follows: alanine (Ala or A), arginine (Arg or R), asparagine (Asn or N), aspartic acid (Asp or D), cysteine (Cys or C), glutamine (Gin or Q), glutamic acid (Glu or E), glycine (Gly or U), histidine (His or II), isoleucine (Ile or I), leucine (Leu or L), lysine (Lys or K), methionine (Met or phenylalanine (Phe or F), proline (Pro or P), serine (Ser or S), threonine (Thr or T), tryptophan (Trp or W), tyrosine (Tyr or Y), and valine (Val or V).

Amino acids are typically organic acids, which substituted or unsubstituted amino groups, substituted or unsubstituted carboxyl groups, and one or more side chains or groups, or any analogs of these groups. Exemplary side chains include, for example, thiol, seleno, sulfonyl, alkyl, aryl, acyl, keto, azido, hydroxyl, hydrazine, cyano, halo, hydrazide, alkenyl, alkynyl, ether, borate, boronate, phospho, phosphono, phosphine, heterocyclic, enone, imine, aldehyde, ester, thioacid, hydroxylamine, or any combination thereof.

Other exemplary amino acids include the following amino acids, but the present invention is not limited thereto: an amino acid including a photoactivatable crosslinking agent, a metal-binding amino acid, a spin-labeled amino acid, a fluorescent amino acid, a metal-containing amino acid, a novel functional group-containing amino acid, an amino acid covalently or non-covalently interacting with another molecule, a photocaged and/or photoisomerizabie amino acid, a radioactive amino acid, an amino acid including a biotin or biotin analog, a glycosylated amino acid, an amino acid modified with another carbohydrate, an amino acid including polyethylene glycol or polyether, a heavy atom-substituted amino acid, chemodegradable and/or photodegradable amino acid(s), a carbon-linked sugar-containing amino acid, a redox-active amino acid, an amino thioacid-containing amino acid, and an amino acid including one or more toxic parts.

Regarding the DNA polymerase of the present invention, the term “mutant” means a recombinant polypeptide including one or more amino acid substitutions, compared to a corresponding naturally-occurring or unmodified DNA polymerase.

The term “thermostable polymerase (referring to a thermostable enzyme)” has thermal resistance, has sufficient activity to achieve subsequent polynucleotide extension, and is not irreversibly denature (inactivated) when treated at elevated temperatures for the time required to achieve the denaturation of a double-stranded nucleic acid. As used herein, it is suitable for a reaction such as PCR to be used at a cycling temperature. Herein, irreversible denaturation refers to the permanent and complete loss of enzyme activity. The enzyme activity of the thermostable polymerase refers to the catalysis of a nucleotide combination by a method suitable for the formation of a polynucleotide extension product which is complementary to a template nucleic acid strand. Thermophilic bacteria-derived thermostable DNA polymerases include, for example, DNA polymerases derived from Hermotoga maritima, Thermus aquaticus, Thermus thermophilus, Thermus flabus, Thermodipyliporphis, Thermus sp. Sps17 , Thermus sp. Z05, Thermus caldophilus, Bacillus caldotenax, Thermotoga neopolitanica and Thermosipo africanus.

The term “thermoactive” refers to an maintaining a catalytic property at temperatures (i.e., 45 to 80° C.) conventionally used in reverse transcription or annealing/extension steps in RT-PCR and/or PCR reactions. The thermostable enzyme is not irreversibly inactivated or denatured when treated at elevated temperatures required for nucleic acid denaturation. The thermoactive enzyme may be thermostable or may not thermostable. The thermoactive DNA polymerase may include, but not limited to, DNA or RNA dependent on thermophilic or mesophilic species.

The term “host cell” includes single-cellular prokaryotic and eukaryotic organisms (e.g., bacteria, yeast, and actinomycetes) and single cells derived from higher plant, an animal, or both thereof.

The term “vector” refers to a DNA molecule which is replicable and able to deliver foreign DNA such as a gene to a recipient cell, for example, a plasmid, a phage, or an artificial chromosome. The “plasmid,” “vector,” or “plasmid vector” used herein may be used interchangeably.

The term “nucleotide” may be a deoxyribonucleic acid (DNA) or a ribonucleic acid (RNA), which is present in a single strand or double strand, and unless particularly described otherwise, an analog of a natural nucleotide may be included.

The term “nucleic acid” or “polynucleotide” refers to a DNA or RNA polymer, or a polymer that can correspond to an analog thereof. The nucleic acid may be, for example, a chromosome or chromosome fragment a vector (e.g., an expression vector), an expression cassette, a naked DNA or RNA polymer, a product of a polymerase chain reaction (PCR), an oligonucleotide, a probe, or a primer, but the present invention is not limited thereto. The nucleic acid may be, for example, a single-stranded, double-stranded, or triple-stranded, but is not limited to any specific length. Unless particularly defined otherwise, a specific nucleic acid sequence includes a complementary sequence in addition to a random sequence noted herein, or encodes the same.

The “primer” refers to a polynucleotide that can serve as a starting point of nucleic acid synthesis in a template-direction under the conditions for the initiation of the extension of a polynucleotide. Primers may also be used in the process of synthesis mediated by various other oligonucleotides which are included as initiators of de novo RNA synthesis and an in vitro transcription-related process. Primers are typically single-stranded oligonucleotides oligodeoxyribonucleotides). The suitable length of a primer varies typically in the range from 6 to 40 nucleotides, and more typically, 15 to 35 nucleotides, according to the intended use. A short primer molecule generally requires a lower temperature to form a sufficiently stable hybridization complex with a template. A primer is not required to correspond to the exact sequence of a template, but needs to be sufficiently complementary to be hybridized with the template subject to extension. In a specific exemplary embodiment, the term “primer pair” means a primer set comprising a 5′-sense primer which is complementarily hybridized to the 5′ end of a nucleic acid sequence to be amplified, and a 3′ -antisense primer which is hybridized to the 3′ end of the sequence to be amplified. A primer may be labeled, if necessary, by being mixed with a marker to be detected by spectroscopic, photochemical, biochemical, immunochemical or chemical means. For example, a useful marker is as follows: 32P, a fluorescent dye, an electron-dense reagent, an enzyme (conventionally used in ELISA), biotin, or a protein that can be used with hapten and an anti-serum or monoclonal antibody.

The term “5′-nuclease probe” refers to an oligonucleotide having one or more luminescent markers which are used in a 5′-nuclease reaction for targeting nucleic acid detection. In some exemplary embodiments, for example, a 5′-nuclease probe only has a single luminescent part (e.g., a fluorescent dye or the like). In a specific exemplary embodiment, a 5-nuclease probe has a self-complementary region to form a hairpin structure under selective conditions. In some exemplary embodiments, a 5′ nuclease probe has two or more markers, and one of the two markers is separated or degraded from the oligonucleotide and then released with an increased radiation intensity. In a specific exemplary embodiment, a 5′-nuclease probe is labeled with two different fluorescent dyes, for example, a 5′ -end reporter dye and a 3′-end quencher dye. In some exemplary embodiments, a 5′-nuclease probe is labeled at one or more positions in addition to or other than the ends. When the probe is intact, typically, energy transfer occurs between two fluorescent materials to partially or completely quench fluorescence emitted from a reporter dye. During extension in PCR, for example, a 5′-nuclease probe binding to a template nucleic acid is degraded by the activity of no longer quenching the fluorescence emission of a reporter dye, for example, the 5′ or 3′-nuclease activity of Taq polymerase or different polymerase. In some exemplary embodiments, a 5′ -nuclease probe may be labeled with two or more different reporter dyes and a 3′-end quencher dye or a part thereof.

The term “FRET” or “fluorescence resonance energy transfer” or “Foerster Resonance Energy Transfer” refers to the transfer of energy between two or more chromophores, donor chromophores and recipient chromophores (referred to as quenchers). Typically, when a donor is excited by radiating light with an appropriate wavelength, energy is transferred to a recipient. The recipient typically re-radiates energy transferred in the form of light radiated with a different wavelength. When the recipient is a “dark” quencher, it disperses energy transferred in a form other than light.

Whether a specific fluorescent material serves as a donor or recipient is dependent on the properties of other members of the FRET pair. Conventionally used donor-recipient pairs include a FAM-TAMRA pair. Conventionally used quenchers are DABCYL and TAMRA. Conventionally used dark quenchers are as follows: BlackHole Quenchers (BHQ), (Biosearch Technologies, Inc., Novato, Cal.), Iowa Black (Integrated DNA Tech., Inc., Coralville, Iowa), and BlackBerry Quencher 650 (BBQ-650) (Berry & Assoc., Dexter, Mich.).

The term “conventional” or “natural” used to describe a nucleic acid base, a nucleoside triphosphate or a nucleotide refers to those naturally occurring in the polynucleotides described herein (i.e., for DNA, dATP, dGTP, dCTP and dTTP). In addition, dlTP and 7-deaza-dGTP are frequently used instead of dGTP, and may be used instead of dATP in an in vitro DNA synthesis reaction such as sequencing.

The term “unconventional” or “modified” used to describe a nucleic acid base, a nucleoside triphosphate or a nucleotide refers to the modification, derivative or analog of a conventional base, nucleoside or nucleotide, which naturally occurs in a specific polynucleotide. A specific, unconventional nucleotide is modified at the 2′ position of the ribose, compared with conventional dNTP. Therefore, although a nucleotide naturally occurring in RNA is a ribonucleotide (i.e., ATP, GTP, CTP, UTP, and collectively, rNTP), since the nucleotide has a hydroxyl group at the 2′ position of the sugar, compared with dNTP having no hydroxyl group, as used herein, the ribonucleotide is a nucleotide which is not conventionally used as a substrate for a DNA polymerase. As used herein, an unconventional nucleotide includes a compound used as a terminator for nucleic acid sequencing, but the present invention is not limited thereto. An exemplary terminator compound includes a compound having a 2′,3′-dideoxy structure, but the present invention is not limited thereto, and is referred to as a dideoxynucleoside triphosphate. Dideoxynucleoside triphosphates such as ddATP, ddTTP, ddCTP and ddGTP are collectively referred to as ddNTP. Additional examples of terminator compounds include 2′-PO4 analogs of a ribonucleotide. Other unconventional nucleotides include phosphorothioate dNTP ([[α]-S]dNTP), 5′-[α]-borano-dNTP, [α]-methyl-phosphonate dNTP, and ribonucleoside triphosphate (rNTP). An unconventional base may be labeled with a radioactive isotope, such as 32P, 33P, or 35S; a fluorescent marker; a chemoluminescent marker; a bioluminescent marker; a hapten marker such as biotin; or an enzyme marker such as streptavidin or avidin. A fluorescent marker may be a negatively-charged dye such as a fluorescein-family dye, or a neutrally-charged dye such as a rhodamine-family dye, or a positively-charged dye such as a cyanine-family dye. Fluorescein-family dyes include, for example, FAM, HEX, TET, JOE, NAN and ZOE. Rhodamine-family dyes include Texas Red, ROX, R110, R6G, and TAMRA. Various dyes or nucleotides labeled with FAM, HEX, TET, JOE, NAN, ZOE, ROX, R110, R6G, Texas Red and TAMRA are commercially available from Perkin-Elmer (Boston, MA), Applied Biosystems (Foster City, CA), or Invitrogen/Molecular Probes (Eugene, OR). Cyanine-family dyes include Cy2, Cy3, Cy5 and Cy7, and are commercially available from GE Healthcare UK Limited (Amersham Place, Little Chalfont, Buckinghamshire, England).

The term “mismatch discrimination” refers to the ability of a biocatalyst (e.g., an enzyme such as a polymerase, ligase, or the like) to discriminate a fully-complementary sequence from a mismatch-containing sequence when a nucleic acid (e.g., primer or a different oligonucleotide) is extended by attaching (for example, covalently) one or more nucleotides to the nucleic acid in a template-dependent manner. The term “mismatch discrimination” refers to the ability of a biocatalyst to discriminate a fully-complementary sequence from a mismatch-containing (approximately complementary) sequence, that is, an extended nucleic acid (e.g., a primer or different oligonucleotide) has a mismatch in the 3′-end nucleic acid, compared with a nucleic acid-hybridized template. In some exemplary embodiments, an extended nucleic acid includes a mismatch at the 3′ end with respect to a fully-complementary sequence. In some exemplary embodiments, an extended nucleic acid includes a mismatch at the penultimate (N-1) 3′ position and/or at the N-2 position relative to the fully complementary sequence.

Unless defined otherwise, all technical and scientific terms used herein have the same meanings as generally understood by those of ordinary skill in the art.

The present invention relates to a DNA polymerase comprising a Taq polymerase amino acid sequence of SEQ ID NO: 1, the DNA polymerase including

a) a substitution at amino acid residue 507 in the amino acid sequence of SEQ ID NO: 1;

(b) (i) a substitution at amino acid residue 536 in the amino acid sequence of SEQ ID NO: 1,

(ii) a substitution at ammo acid residue 660 in the amino acid sequence of SEQ ID NO: 1,

(iii) substitutions at amino acid residues 536 and 660 in the amino acid sequence of SEQ ID NO: 1, or

iv) substitutions at amino acid residues 536, 587 and 660 in the amino acid sequence of SEQ ID NO:

The “Taq polymerase” is a heat-resistant DNA polymerase named after thermophilic bacteria Thermus aquaticus, and was first isolated from the bacteria. Thermus aquaticus are bacteria living in hot springs and hydrothermal vents, and the Taq polymerase is an enzyme that can tolerate a protein-denaturing condition (high temperature) required during PCR. The Taq polymerase has an optimal activity temperature of 75 to 80° C., has a half-life of 2. hours or more at 92.5° C., 40 minutes at 95° C. and 9 minutes at 97.5° C., and can replicate 1000-bp DNA within 10 seconds at 72° C. It lacks 3′-5′ exonuclease proofreading activity, resulting in an error rate of approximately 1 in 9,000 nucleotides. For example, when heat-resistant Taq is used, PCR may be performed at a high temperature (60° C. or more). The amino acid sequence set forth in SEQ ID NO: 1 is used as a reference sequence for the Taq polymerase.

According to an exemplary embodiment of the present invention, the substitution at amino acid residue 507 is a substitution of glutamic acid (E) with lysine (K), the substitution at amino acid residue 536 is a substitution of arginine (R) with lysine (K), the substitution at amino acid residue 587 is a substitution of arginine (R) with isoleucine (1), and the substitution at amino acid residue 660 may be a substitution of arginine (R) with valine (V).

In the present invention, the Taq polymerase in which glutamic acid (E) substituted with lysine (K) at the amino acid residue 507 in the amino acid sequence of SEQ ID NO: 1 is named “E507K” (SEQ ID NO: 2); the Taq polymerase in which glutamic acid (E) is substituted with lysine (K) at the amino acid residue 507, and arginine (R) is substituted with lysine (K) at the amino acid residue 536 in the amino acid sequence of SEQ ID NO: I is named “E507K/R536K” (SEQ ID NO: 6); the Taq polymerase in which glutamic acid (E) is substituted with lysine (K) at the amino acid residue 507, and arginine (R) is substituted with valine (V) at the amino acid residue 660 in the amino acid sequence of SEQ ID NO: 1 is named “E507K/R660V” (SEQ ID NO: 7); the Taq polymerase in which glutamic acid (E) is substituted with lysine (K) at the amino acid residue 507, arginine (R) is substituted with lysine (K) at the amino acid residue 536, and arginine (R) is substituted with valine (V) at the amino acid residue 660 in the amino acid sequence of SEQ ID NO: 1 is named “E507K/R536K/R660V” (SEQ ID NO: 8); and finally the Taq polymerase in which glutamic acid (E) is substituted with lysine (K) at the amino acid residue 507, arginine (R) is substituted with lysine (K) at the amino acid residue 536, arginine (R) is substituted with isoleucine (1) at the amino acid residue 587, and arginine (R) is substituted with valine (V) at the amino acid residue 660 in the amino acid sequence of SEQ ID NO: 1 is named “E507K/R536K/R587I/R660V” (SEQ NO: 37).

According to an exemplary embodiment of the present invention, the DNA polymerase discriminates a matched primer from a mismatched primer, the matched primer and the mismatched primer are hybridized with a target sequence, and the mismatched primer may include a non-canonical nucleotide at the 3′ end with respect to a hybridized target sequence.

The mismatched primer is a hybrid oligonucleotide which should be sufficiently complementary to be hybridized with the target sequence, but does not correspond to the exact sequence of the target sequence.

The “canonical nucleotide” or “complementary nucleotide” means a standard Watson-Crick base pair, A-U, A-T or G-C.

The “non-canonical nucleotide” or “non-complementary nucleotide” means A-C, A-G, G-U, G-T, T-C, T-U, A-A, G-G, T-T, U-U, C-C, or C-U other than the Watson-Crick base pairs.

According to an exemplary embodiment of the present invention, with the DNA polymerase, the amplification of a target sequence including a matched primer may exhibit a lower Ct value than the amplification of a target sequence including a mismatched primer.

For example, the DNA polymerase may allow one or more nucleotides to covalently bind to a primer, thereby extending a matched primer with greater efficiency than a mismatched primer in a target sequence-dependent manner. Here, greater efficiency may be observed at a lower Ct value for a matched primer, compared with the mismatched primer, for example, in RT-PCR. The difference in Ct value between the matched primer and the mismatched primer may be 10 or more, and preferably, 10 to 20, or there may be no synthesis of an amplicon by a mismatched primer.

For example, such a difference means that a product formed by standard PCR using a forward primer and a reverse primer, which are matched in a first reaction, and a reverse primer matched with a mismatched forward primer in a second reaction with the same experiment settings is larger in the first reaction than in the second reaction.

A Ct (threshold crossing cycle) value represents a DNA quantification method by quantitative PCR, which depends on plotting the fluorescence representing the number of cycles on a log scale. The threshold for DNA-based fluorescence detection is set slightly higher than the minimum background. The number of cycles required for fluorescence to cross the threshold is called Ct or a quantification cycle (Cq) following the MIQE guidelines. The Ct value for the given reaction is defined as the number of cycles required for fluorescence emission to cross a fixed threshold. For example, SYBR Green I and a fluorescent probe may be used in real-time PCR for template DNA quantification. Fluorescence emitted from a sample is collected every cycle during PCR, and plotted against the number of cycles. A starting template concentration is inversely proportional to the time at which the fluorescent signal is first shown. The signal appears earlier as a template concentration is higher (shown at a low number of cycles).

The present invention also relates to a nucleic acid sequence encoding the above-described DNA polymerase, and a vector and a host cell, which include the nucleic acid sequence. Various vectors may be prepared using the nucleic acid encoding the DNA polymerase of the present invention. Any vector having a replicon and a control sequence, which are derived from a species compatible with a host cell, may be used. The vector of the present invention may be an expression vector, and has nucleic acid regions for regulating transcription and translation, which are operably linked to the nucleic acid sequence encoding the DNA polymerase of the present invention. The regulatory sequence refers to a DNA sequence required for the expression of a coding sequence operably linked to a specific host organism. For example, a control sequence suitable for a prokaryote includes a promoter, any operating sequence and a ribosome binding sequence. In addition, a vector may include a “positive retroregulatory element (PRE)” to increase the half-life of mRNA to be transcribed. Transcription and translation regulatory nucleic acid regions may be generally suitable for host cells used to express a polymerase. Various types of suitable expression vectors and regulatory sequences are known to be used for various host cells. Generally, transcription and translation regulatory sequences may include, for example, a promoter sequence, a ribosome binding site, transcription initiation and termination sequences, translation initiation and termination sequences, and an enhancer or activation sequence. In a typical, exemplary embodiment, regulatory sequences include a promoter and transcription initiation and termination sequences. Typically, a vector also includes a polylinker region containing several restriction sites for inserting foreign DNA, In a specific exemplary embodiment, the “fusion flag” is used to promote purification, and if necessary, a tag/flag is subsequently removed (e.g., “His-Tag”). However, when thermoactive and/or thermostable protein(s) is(are) purified from mesophilic hosts (e.g., E. coil) using a “heating step,” the fusion flags are generally unnecessary. A suitable vector containing a DNA encoding replication sequence, a regulatory sequence and a phenotype selection gene is constructed, and a mutant polymerase of interest is prepared using a standard recombinant DNA technique. An isolated plasmid, a viral vector and a DNA fragment are digested and cleaved, and then ligated with each other in a specific order to form a desired vector as known in the art.

In an exemplary embodiment of the present invention, an expression vector contains a selectable marker gene to select a transformed host cell. Selection genes are known in the art, and may vary according to the host cells used herein. Suitable selection genes may include the gene coding for ampicillin and/or tetracycline resistance, and may allow cells in which these vectors are cultured in the presence of these antibiotics to be transformed.

In an exemplary embodiment of the present, invention, a nucleic acid sequence encoding the DNA polymerase of the present invention may be introduced into cells alone or in combination with a vector. The introduction or equivalent expressions thereof refer to a nucleic acid entering cells in the method suitable for subsequent integration, amplification and/or expression. The introduction method includes, for example, CaPO₄ precipitation, liposome fusion, LIPOFECTIN®, electrophoresis, and viral infection.

Prokaryotes are used as host cells in an early cloning step of the present invention. They are particularly useful for rapidly preparing a great quantity of DNA, for preparing a single-stranded DNA template used in site-directed mutagenesis, for simultaneously screening many mutants, and for DNA sequencing of generated mutants. Suitable prokaryotic host cells include E. coli K12 strain 94 (ATCC No. 31,446), E. coil strain W3110 (ATCC No. 27,325), E. coli K12 strain DG116 (ATCC No. 53,606), E. coli X1776 (ATCC No. 31,537), and E. coli B; many other strains of E. coli, such as HB101, JM101, NM522, NM538 and NM539, and other species such as Bacilli, e.g., Bacillus subtilis, other Enterobacteriaceae, e.g., Salmonella typhimurium or Serratia marcescens, and prokaryotic genera including various Pseudomonas sp. may be used as hosts. Typically, plasmids used in the transformation of E. coil include pBR322, pUCI8, pUCI9, pUCI18, pUC119 and Bluescript M13. However, many other suitable vectors may also be used.

The present invention also provides a method of preparing a DNA polymerase, which includes: culturing the host cells; and isolating a DNA polymerase from a cell culture and a supernatant thereof.

The DNA polymerase of the present invention is prepared by culturing host cells transformed with an expression vector containing a nucleic acid sequence encoding the DNA polymerase under suitable conditions inducing or causing the expression of the DNA polymerase. A method of culturing the transformed host cells under conditions suitable for protein expression is known in the art. Host cells suitable for the preparation of a polymerase from a lambda (λ) pL, promoter-containing plasmid vector include E. coli strain DG116 (ATCC No. 53606). When expressed, the polymerase may be collected and isolated.

After purification, mismatch discrimination of the DNA polymerase of the present invention may be assayed. For example, mismatch discrimination activity is measured by comparing the amplification of a target sequence perfectly matched with a primer with respect to the amplification of a target having a single base mismatch at the 3′ end of a primer. The amplification may be detected in real time by using, for example, a TaqMan™ probe. The ability of a polymerase to distinguish between two target sequences may be assumed by comparing Cts in two reactions.

Therefore, the present invention provides a method of in vitro detecting one or more gene variations or SNPs in one or more templates, the method including:

bringing the DNA polymerase according to the present, invention into contact with a) one or more templates,

b) a nucleoside triphosphate, and

c) one or more matched primers, one or more mismatched primers or both of one or more matched primers and one or more mismatched primers, wherein the one or more matched primers and the one or more mismatched primers are hybridized with a target sequence, and the mismatched primer has a non-canonical nucleotide at base position 7 from the 3′ end thereof with respect to the hybridized target sequence.

The “single-nucleotide polymorphism (SNP)” refers to a genetic change or variation showing the difference of a single base (A, T, G or C) in a DNA base sequence.

In the method of in vitro detecting a gene variation or SNP, a target sequence may be present in a test sample, including, for example, DNA, cDNA or RNA, and preferably, genomic DNA. The test sample may be a cell lysate prepared from bacteria, a bacterial culture, or a cell culture. In addition, the test sample may be one included in an animal, preferably, a vertebrate, and more preferably, a human subject. The target sequence may be genomic DNA, preferably, genomic DNA of an individual, more preferably, bacteria or a vertebrate, and most preferably, genomic DNA of a human subject.

The SNP detection method of the present invention may include analysis of a melting temperature using a double strand-specific dye such as SYBR Green I.

The analysis of a melting temperature curve may be performed in a real-time PCR instrument such as ABI 5700/7000 (96-well format) or ABI 7900 (384-well format) instrument with onboard software (SDS 2.1). Alternatively, the analysis of a melting temperature curve may be performed as end-point analysis.

The “dye binding to double-stranded DNA” or “double strand-specific dye” may be used when high fluorescence is emitted while binding to double-stranded DNA, rather than in an unbound state. Examples of these dyes are SOYTO-9, SOYTO-13, SOYTO-16, SOYTO-60, SOYTO-64, SYTO-82, ethidium bromide (EtBr), SYTOX Orange, TO-PRO-1, SYBR Green I, TO-PRO-3 or EvaGreen. These dyes excluding EtBr and EvaGreen (Qiagen) have been tested in real-time applications.

The method of in vitro detecting a gene variation or SNP may be performed by real-time PCR, analysis on agarose gel after standard PCR, gene variation-specific amplification or allele-specific amplification through real-time PCR, tetra-primer amplification-refractory mutation system PCR or isothermal amplification, but the present invention is not limited thereto.

For example, the SNP detection method of the present invention may be performed using sequencing, mini-sequencing, allele-specific PCR, dynamic allele-specific hybridization (DASH), a PCR extension assay (e.g., single base extension SBE), PCR-SSCP, a PCR-KELP assay or TaqMan method, SNPlex platform (Applied Biosystems), mass spectrometry (e.g., MassARRAY system of Sequenom), or a .Bio-Plex system. (BioRad).

The “standard PCR” is a technique for amplifying single or several copies of DNA or cDNA known to a technician of ordinary skill in the art. Almost all PCR techniques use a thermostable DNA polymerase such as Taq polymerase or Klen Taq. A DNA polymerase uses single-stranded DNA as a template, and enzymatically assembles a new DNA strand from nucleotides using oligonucleotide primers. Amplicons generated by PCR may be analyzed on, for example, agarose gel.

The “real-time PCR” may monitor a PCR process in real time. Therefore, data is collected throughout the PCR process, not at the end of PCR. In the real-time PCR, the reaction is characterized by the point of time during a cycle when amplification is first detected, rather than the amount of a target accumulated after a fixed number of cycles. Usually, both of dye-based detection and probe-based detection are used to perform quantitative PCR.

The “allele-specific amplification (ASA)” is an amplification technique for designing PCR primers to discriminate templates with different single nucleotide residues.

The “allele-specific amplification or gene variation-specific amplification through real-time PCR” is a highly effective method for detecting a gene variation or SNP. Unlike most of other methods for detecting a gene variation or SNP, the pre-amplification of a target gene material is not needed. ASA combines amplification and detection in a single reaction based on the discrimination between matched and mismatched primer/target sequence complex. The increase in amplified DNA during the reaction may be monitored in real time with the increase in fluorescent signal caused by a dye such as SYBR Green I emitted upon binding to double-stranded DNA. The allele-specific amplification or gene variation-specific amplification through real-time PCR shows the delay or absence of a fluorescent signal when a primer is mismatched. In the gene variation or SNP detection, such amplification provides information on the presence or absence of a gene variation or SNP.

The “tetra-primer amplification-refractory mutation system PCR” is amplification of all of wild-type and mutant alleles with a control fragment in single tube PCR. A non-allele-specific control amplicon is amplified by two common (outside) primers flanking a mutation region. The two allele-specific (inside) primers are designed in an opposite direction to the common primers, both of wild-type and mutant amplicons may be simultaneously amplified with the common primers. As a result, two allele-specific amplicons may have different lengths since mutations are asymmetrically located based on the common (outside) primers, and easily separated by standard gel electrophoresis. The control amplicons provide an internal control for false negative results as well as amplification failure, and at least one of the two allele-specific amplicons is always present in the tetra-primer amplification-refractory mutation system PCR.

The “isothermal amplification” means that the amplification of a nucleic acid is not dependent on a thermocycler and is performed at a lower temperature without the need for temperature change during amplification. The temperature used in isothermal amplification may range from room temperature (22 to 24° C.) to approximately 65° C., or approximately 60 to 65° C., 45 to 50° C., 37 to 42° C. or room temperature (22 to 24° C.). A product obtained by the isothermal amplification may be detected by gel electrophoresis, ELISA, enzyme-linked oligosorbent assay (ELOSA), real-time PCR, enhanced chemiluminescence (ECL), a chip-based capillary electrophoresis device, such as a bioanalyzer, for analyzing RNA, DNA and protein or turbidity.

In an exemplary embodiment of the present, invention, E507K/R536K, E507K/R660V, or E507K/R536K/R660V Taq polymerase was used to confirm whether an ability of extending a mismatched primer with respect to a template including a SNP (rs1408799, rs1015362 and/or rs4911414) was reduced.

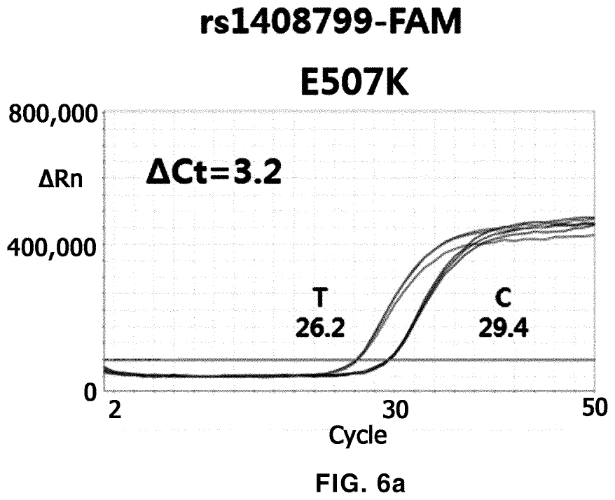

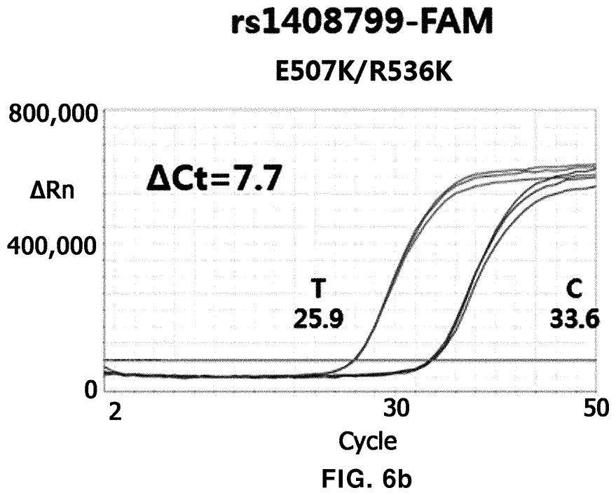

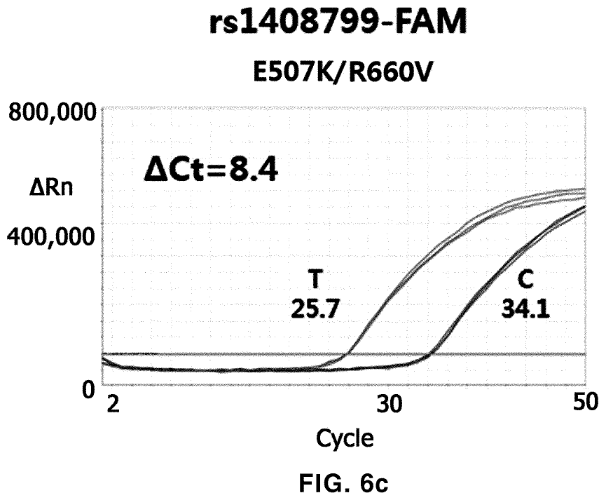

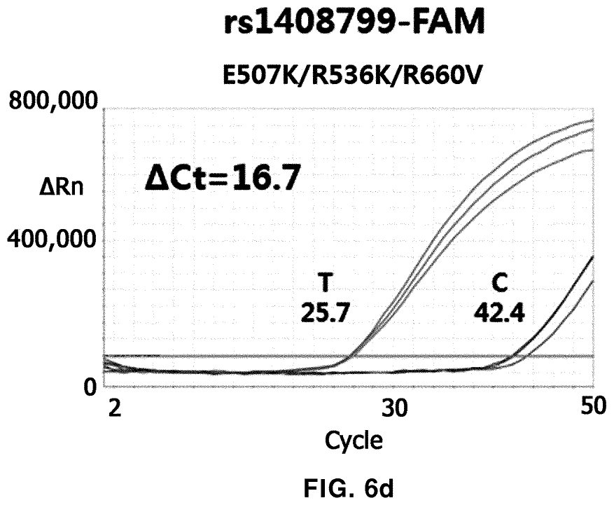

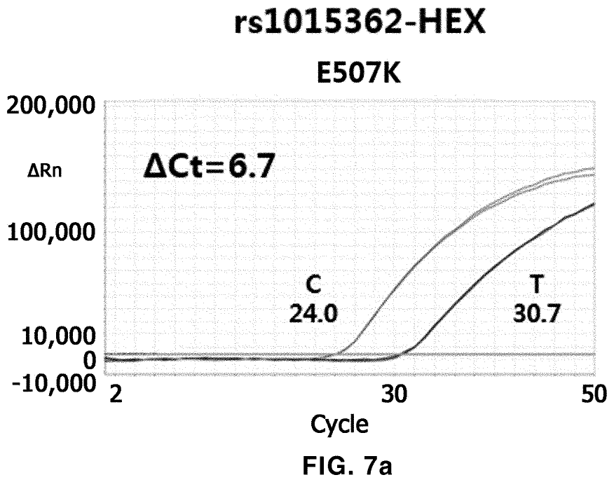

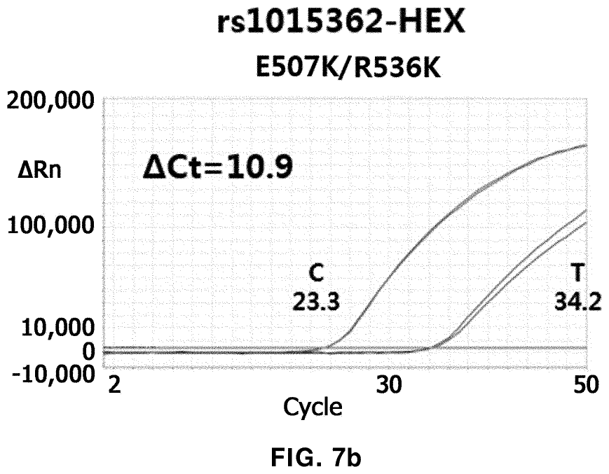

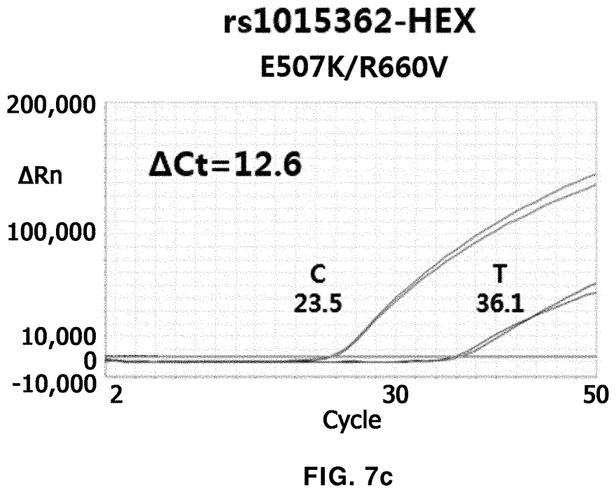

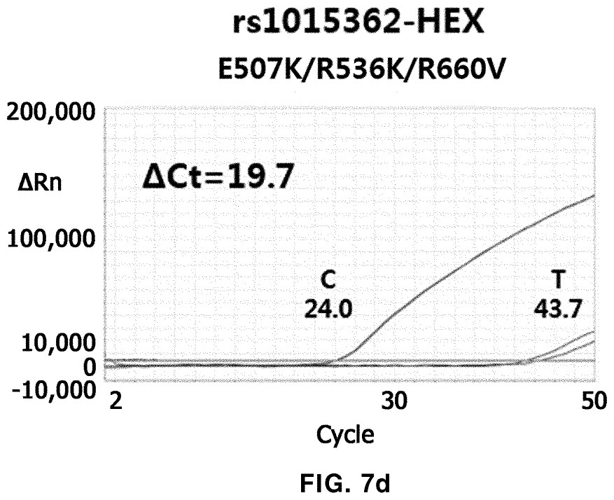

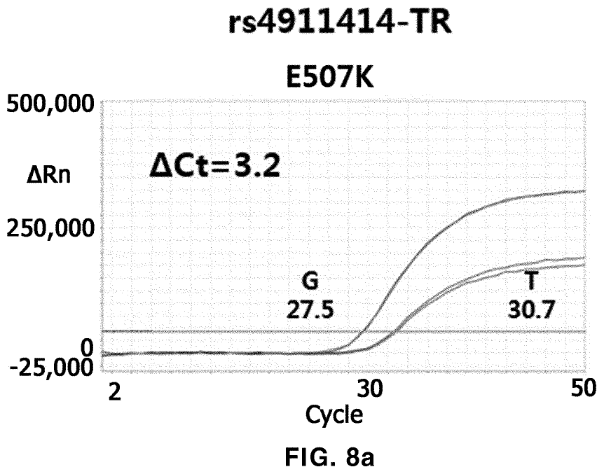

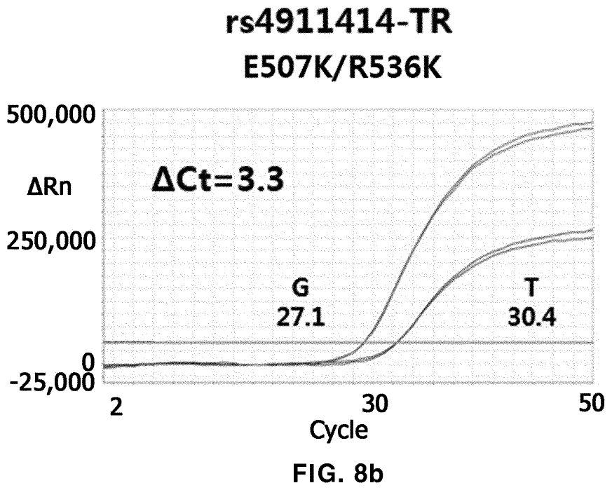

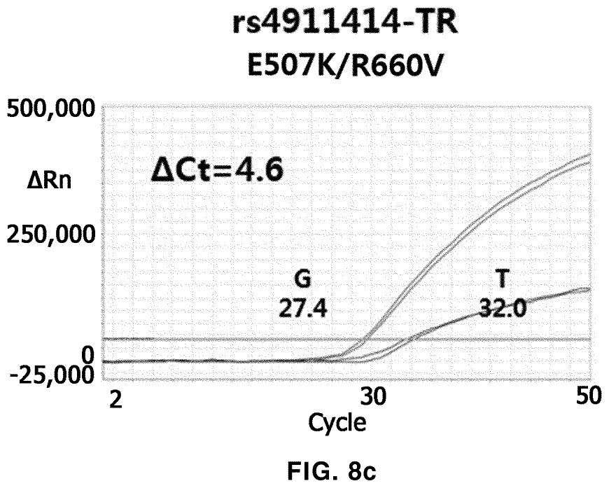

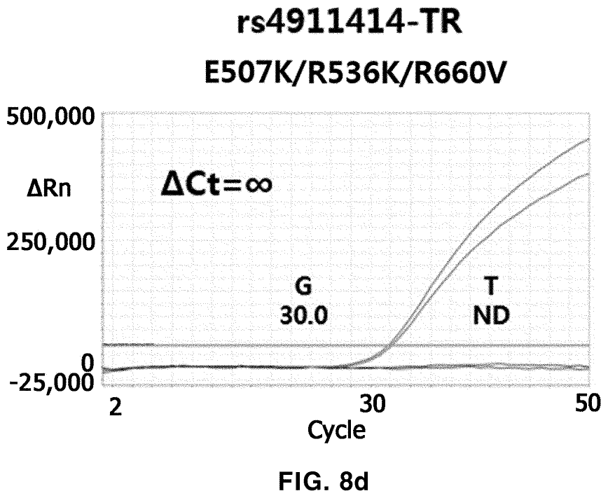

As a result, as shown in FIGS. 6a-6d, 7 and 8, compared to E507K Taq polymerase, in the case of the E507K/R536K, E507K/R660V or E507K/R536K/R660V Taq polymerase, it can be confirmed that amplification with a mismatched primer is delayed, and such an effect was most clearly shown in the case of the E507K/R536K/R660V Taq polymerase.

To this end, it was confirmed that the three types of DNA polymerases have higher mismatch extension selectivity than the conventional Taq polymerase (E507K). Therefore, it is expected that the DNA polymerase of the present invention can be effectively used in medical diagnosis of a disease and recombinant DNA studies.

In another exemplary embodiment of the present invention, the E507K/R536K/R/R660V Taq polymerase was used to confirm whether an ability of extending mismatched primers with respect to a template with Q61H, G13D or GUS SNP at the KRAS gene, and a template with L858R SNP at the EGFR gene was reduced.

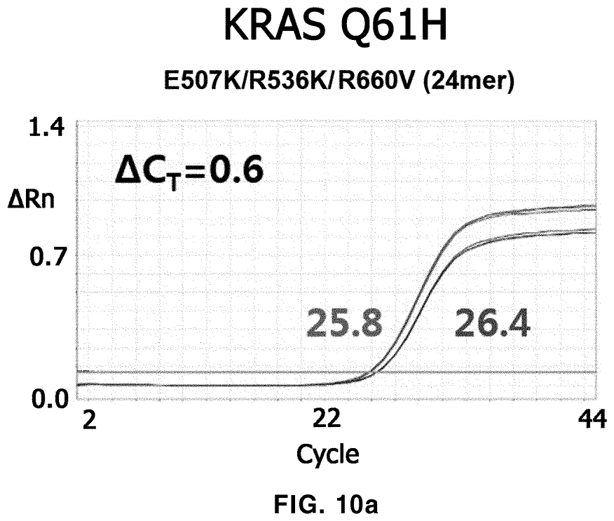

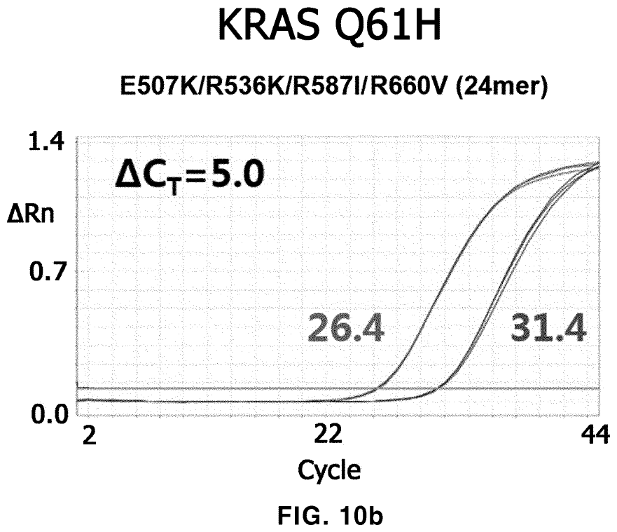

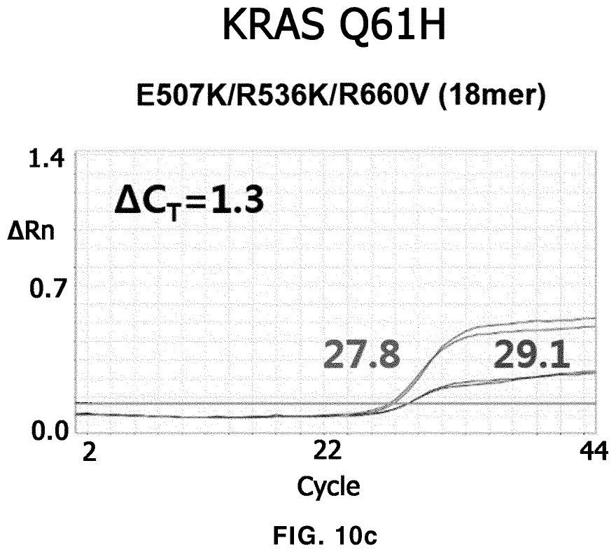

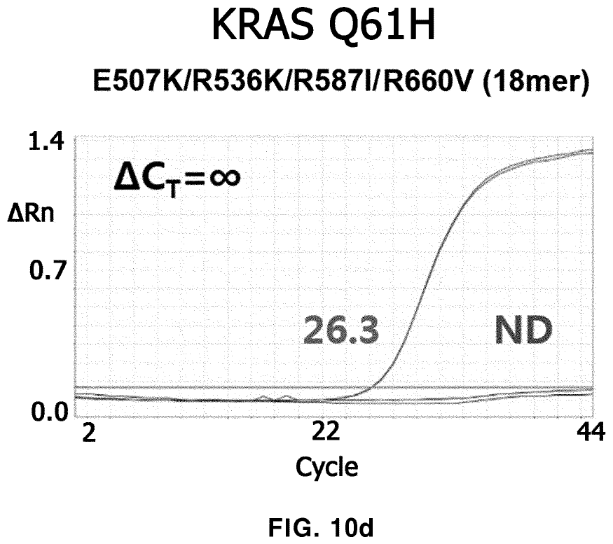

As a result, as shown in FIGS. 10a to 10d, 11, 12 and 13. it was confirmed that Taq DNA polymerase having E507K/R536K/R587I/R660V variations, compared with Taq polymerase having E507K/R536K/R660V variations, has superior mismatch extension selectivity. Therefore, it is expected that the Taq DNA polymerase having E507K/R536K/R587I/R660V variations according to the present invention can also be effectively used in medical diagnosis of a disease and recombinant DNA studies.

The present invention also relates to a composition for detecting a gene variation or SNP, which includes the DNA polymerase according to the present invention, and a PCR kit including the same.

According to an exemplary embodiment of the present invention, the PCR kit may be applied to general PCR (first generation PCR), real-time PCR (second generation PCR), digital PCR (third generation PCR) or MassARRAY.

In the PCR kit of the present invention, the digital PCR may be competitive allele-specific TaqMan PCR (CAST PCR) or droplet digital PCR (ddPCR), and more specifically, allele-specific cast PCR or allele-specific droplet digital PCR, but the present invention is not limited thereto.

The “CAST PCR” is a method of detecting and quantifying rare mutations from a large amount of sample containing normal wild-type gDNA, and to inhibit non-specific amplification from a wild-type allele, higher specificity may be generated by the combination of allele-specific TaqMan® qPCR with an allele-specific MGB inhibitor, compared to traditional allele-specific PCR.

The “droplet digital PCR” is a system for counting target DNA after a 20 μl PCR product is fractionated into 20,000 droplets and then amplified, and may be used to count positive droplets (1) and negative droplets (0) considered as digital signals according to the amplification of target DNA in droplets, calculate the number of copies of target DNA by the Poisson distribution, and finally determine result values with the number of copies per μl sample, and used to detect rare mutations, amplify a very small amount of gene and simultaneously confirm a mutation type.

The “MassARRAY” is a multiplexing analysis method that can be applied to various genome studies such as genotyping, using a MALDI-TOF mass spectrometer, and may be used to rapidly analyze various samples and targets at low cost or to perform customized analysis only for a specific target.

The PCR kit of the present invention may include any reagent or other elements, which are recognized for use in primer extension by technicians of ordinary skill in the art.

According to an exemplary embodiment of the present invention, the PCR kit may further include one or more matched primers, one or more mismatched primers or both of one or more matched primers and one or more mismatched primers, wherein the one or more matched primers and one or more mismatched primers are hybridized with a target sequence, and the mismatched primer may include a non-canonical nucleotide at a position of 7 bases from the 3′ end of the primer with respect to the hybridized target sequence.

The PCR kit of the present invention may further include a nucleoside triphosphate.

The PCR kit of the present invention may further include a) one or more buffers; b) a quantification reagent binding to double-stranded DNA; c) a polymerase blocking antibody; d) one or more control values or control sequences; and e) one or more templates.

The present invention relates to a PCR buffer composition for increasing the activity of a DNA polymerase with increased gene variation specificity, which includes 25 to 100 mM KCl; and 1 to 15 mM (NH₄)₂SO₄, and has the final pH of 8.0 to 9.0.

Polymerases used in PCR should be optimal reaction buffers mixed with various materials to perform proper functions. The reaction buffers generally contain an element for pH stabilization, a metal ion as a cofactor, and a stabilization element for preventing the denaturation of a polymerase.

The KCl is an element required for enzyme stabilization, and helps pairing of a primer to target DNA. In the present invention, an optimal concentration was determined by adjusting a KCl concentration in the reaction buffer to confirm a cation concentration in a state in which the, amplification by mismatching is delayed as much as possible, and the amplification efficiency by matching is not reduced.

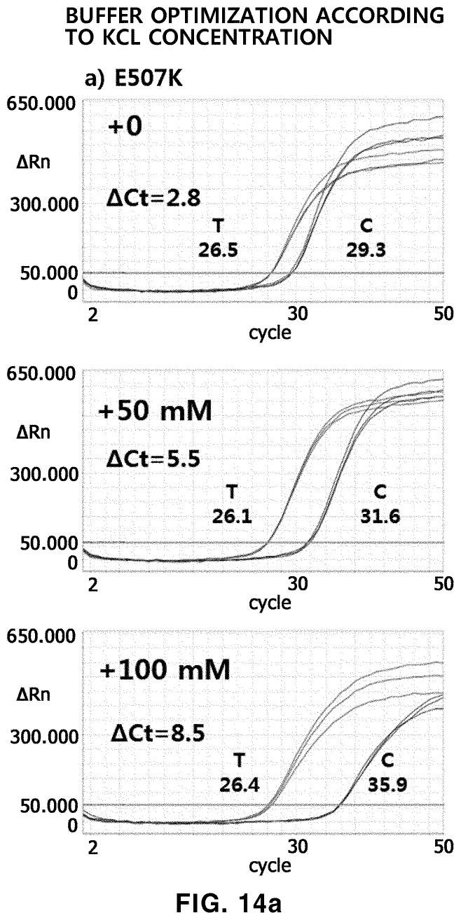

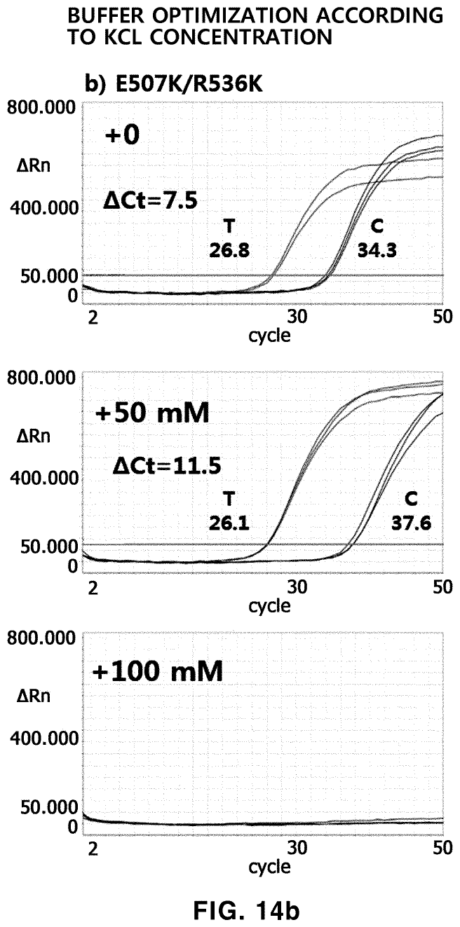

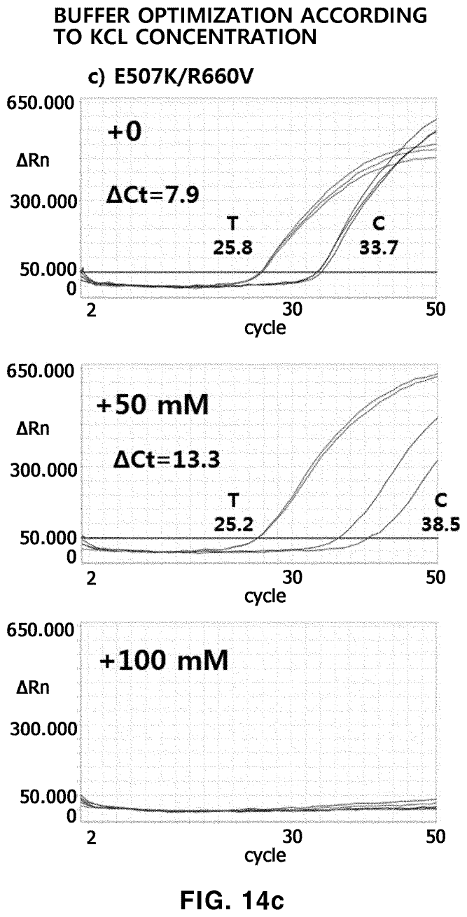

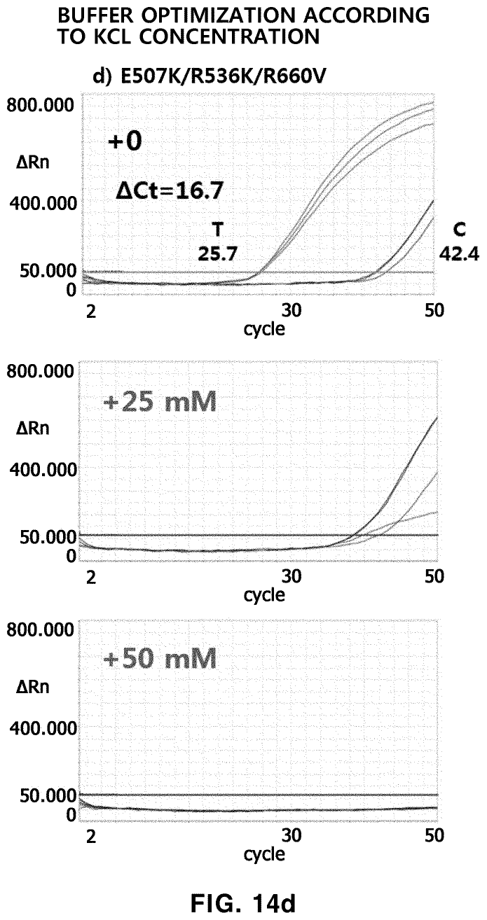

As a result of confirming an amplification delay effect by mismatching according to the change in KCl concentration in the reaction buffer using each of E507K, E507K/R536K, E507K/R660V and E507K/R536K/R660V Taq polymerases, as shown in FIGS. 14a to 14d, the E507K/R536K/R660V Taq polymerase showed an excellent amplification delay effect caused by mismatching without KCl in the reaction buffer, the E507K/R536K and E507K/R660V Taq polymerases showed an excellent amplification delay effect caused by mismatching at 50 mM, and the control E507KTaq polymerase showed an excellent amplification delay effect caused by mismatching at 100 mM. Consequently, it was confirmed that the KCl. concentration threshold is the lowest for the E507K/R536K/R660V Taq polymerase, and lower for the E507K/R536K and E507K/R660V Taq polymerases, compared to that of E507K Taq polymerase.

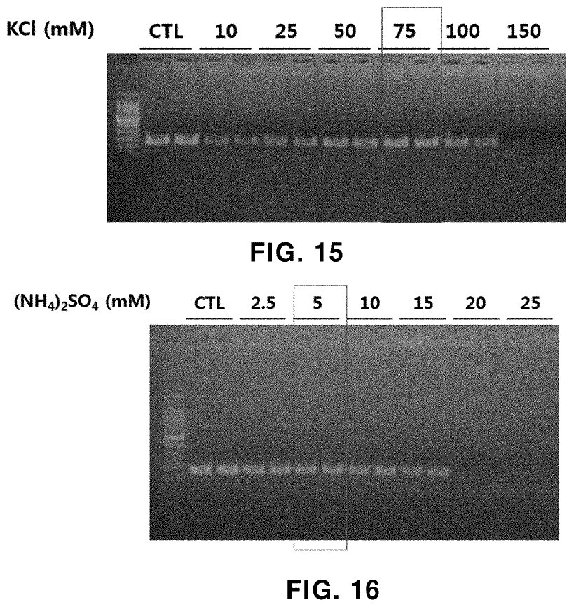

In addition, to determine the optimal KCl concentration, the E507K/R536K/R660V Taq polymerase was used, and amplification was performed by variously changing a KCl concentration while a (NH₄)₂SO₄ concentration was constantly fixed in the reaction buffer. As a result of electrophoresis performed on an amplicon, as shown in FIG. 15, the optimal KCl concentration was confirmed to be 75 mM.

Therefore, the KCl concentration of the PCR buffer composition of the present invention may be 25 to 100 preferably, 60 to 90 more preferably, 70 to 80 mM, and most preferably, 75 mM.

When the KCl concentration is less than 25 mM, it has no influence on general target amplification, but a difference between amplification by a matched primer and amplification by a mismatched primer may be reduced, and when the KCl concentration is more than 100 mM, the efficiency of general target amplification may be lowered.

In the PCR buffer composition, the (NH₄)₂SO₄ is a cofactor required for enzyme activity, and used to increase polymerase activity along with Tris. In an exemplary embodiment of the present invention, based on the determined results, the KCl concentration in the reaction buffer was constantly fixed at 75 mM, and the (NH₄)₂SO₄ concentration varied from 2.5 mM to 25 mM, so that the optimal (NH₄)₂SO₄ concentration was confirmed.

As a result, as shown in FIG. 16, at the (NH₄)₂SO₄ concentrations ranging from 2.5 to 15 mM, an amplicon was identified, confirming that the optimal (N₄)₂SO₄ concentration was 5 mM.

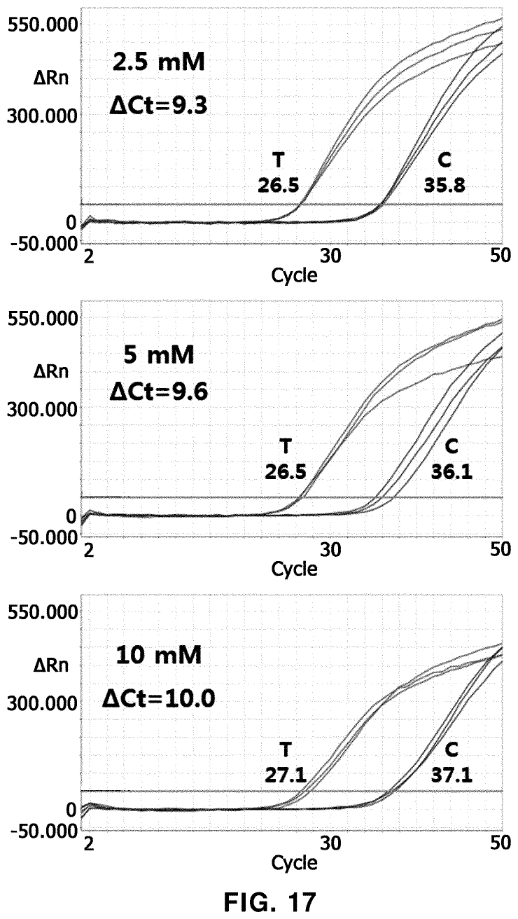

In addition, by performing AS-qPCR at (NH₄)₂SO₄ concentrations of approximately 5 mM (2.5 mM, 5 mM and 10 mM), as shown in FIG. 17, it was confirmed that the Ct difference was the highest at 10 mM, but Ct was a little delayed and a peak was tilted in the amplification caused by matching, and the optimal (NH₄)₂SO₄ concentration was determined to be 5 mM.

Therefore, the (NH₄)₂SO₄ concentration in the PCR buffer composition may be 1 to 15 mM, preferably, 2.5 to 8 mM, more preferably, 4 to 6 mM, and most preferably, 5 mM.

When the (NH₄)₂SO₄ concentration is less than 1 mM, there was no influence on general target amplification, but the difference between the amplification by a matched primer and the amplification by a mismatched primer may be reduced, and when the (NH₄)₂SO₄ concentration is more than 15 mM, the general target amplification efficiency may be lowered.

Therefore, the optimized PCR buffer composition of the present invention may contain 70 to 80 mM KCl and 4 to 6 mM (NH₄)₂SO₄, and the final pH is 8.0 to 9.0.

The PCR buffer composition of the present invention may further include 5 to 80 mM tetra methyl ammonium chloride (TMAC).

TMAC is generally used to reduce amplification caused by mismatching or improve the stringency of a hybridization reaction. In an exemplary embodiment of the present invention, based on the obtained results, the optimal TMAC concentration was determined by fixing the KCl concentration at 75 mM, and the (NH₄)₂SO₄ concentration at 5 mM in the reaction buffer, and varying a TMAC concentration from 0 to 80 mM.

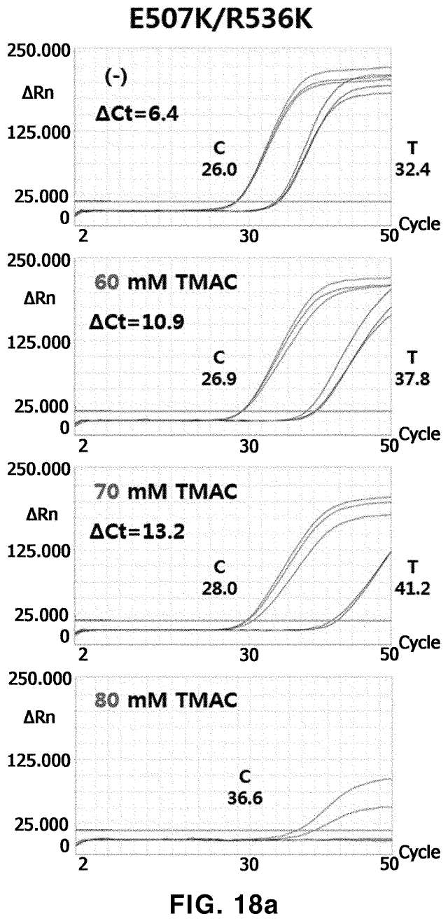

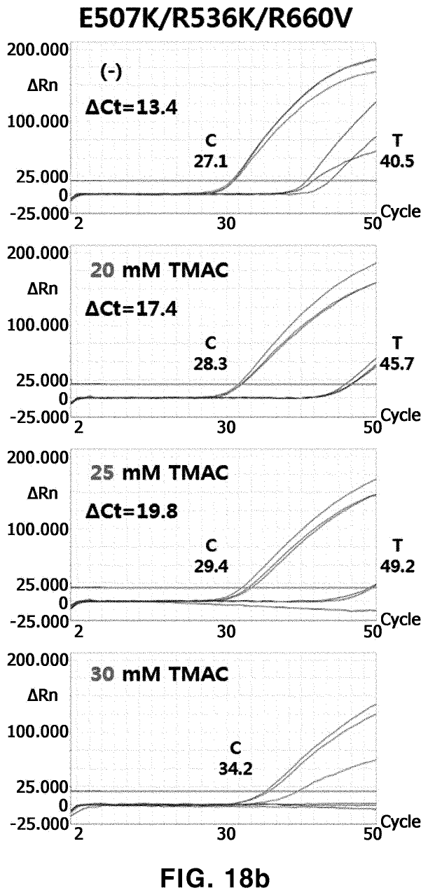

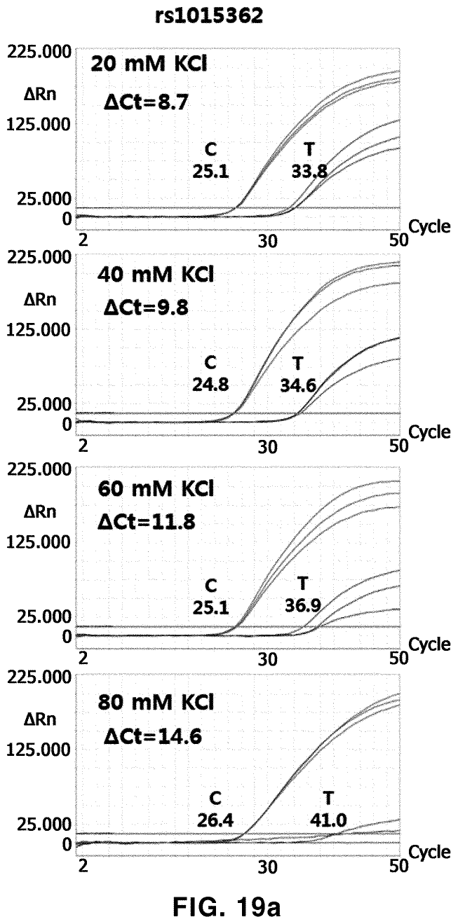

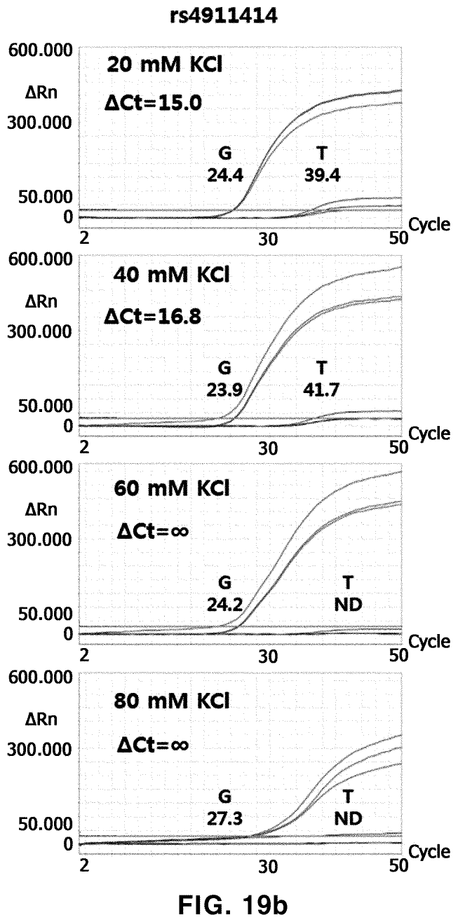

As a result, as shown in FIGS. 18a and 18b, it was confirmed that the optimal TAMC concentration was 70 mM for the E507K/R5361(Taq polymerase, and 25 nM for the E507K/R536K/R660V Taq polymerase. In addition, as a result of amplification performed by constantly fixing a TMAC concentration at 25 mM and a (NH₄)₂SO₄ concentration at 2.5 mM, and varying a KCl concentration to 20, 40, 60 and 80 mM, as shown in FIGS. 19a and 19b, the optimal KCl concentration for SNP rs1015362 and rs4911414 was determined to be 60 mM.

When the TMAC concentration is more than 80 mM, amplification efficiency is reduced, and thus it is preferable that the TMAC concentration is in the above-mentioned range.

Therefore, when the PCR buffer composition of the present invention contains 5 to 80 mM TMAC, the KCl concentration may be 40 to 90 mM, and preferably, 50 to 80 mM, and the (NH₄)₂SO₄ concentration may be 1 to 7 mM, and preferably 1.5 to 6 mM.

When TMAC is contained, the optimized PCR buffer composition of the present invention may contain 15 to 70 nM TMAC, 50 to 80 mM KCl, and 1.5 to 6 mM (NH₄)₂SO₄, and the final pH may be 8.0 to 9.0.

The PCR buffer composition of the present invention may further contain Tris.Cl and MgCl₂, and additionally contain Tween 20 and bovine serum albumin (BSA).

The present invention also provides a PCR kit for detecting a gene variation or SNP, which includes the above-described PCR buffer composition.

The PCR kit of the present invention may further include a DNA polymerase comprising a Taq polymerase amino acid sequence of SEQ ID NO: 1, where the DNA polymerase has substitution(s) of the following amino acids:

(a) a substitution at amino acid residue 507 in the amino acid sequence of SEQ ID NO. 1; and

(b) (i) a substitution at amino acid residue 536 in the amino acid sequence of SEQ ID NO: 1,

(ii) a substitution at amino acid residue 660 in the amino acid sequence of SEQ ID NO: 1,

(iii) substitutions at amino acid residues 536 and 660 in the amino acid sequence of SEQ ID NO: 1, or

(iv) substitutions at amino acid residues 536, 587 and 660 in the amino acid sequence of SEQ ID NO: 1.

According to an exemplary embodiment of the present invention, the substitution at the amino acid residue 507 may be a substitution of glutamic acid (E) with lysine (K), the substitution at the amino acid residue 536 may be a substitution of arginine (R) with lysine (K), the substitution at the amino acid residue 587 may be a substitution of arginine (R) with isoleucine (I), and the substitution at the amino acid residue 660 may be a substitution of arginine (R) with valine (V).

An additional description of the DNA polymerase included in the PCR kit of the present invention is the same as described above, and therefore, the overlapping description will be omitted.

Other components of the PCR kit for detecting a gene variation or SNP, which includes the PCR buffer composition of the present invention are the same as described above, and the overlapping description will be omitted.

The present invention also relates to a method of in vitro detecting one or more gene variations or SNPs using the kit for detecting a gene variation or SNP, which includes the PCR buffer composition for increasing activity of the DNA polymerase with increased gene variation specificity.

The method is the same as the method of in vitro detecting one or more gene variations or SNPs in one or more templates using the DNA polymerase with increased gene variation specificity of the present invention, except the components of the PCR buffer composition, and therefore, the overlapping description will be omitted.

Hereinafter, the present invention will be described in further detail with reference to examples. The examples are merely provided to more fully describe the present invention, and it will be obvious to those of ordinary skill in the art that the scope of the present invention is not limited to the following examples.

### EXAMPLES

### Example 1

Mutagenesis of Taq polymerase

1-1. Fragment PCR

In this example, Taq DNA polymerase in which arginine was substituted with lysine at amino acid residue 536 in the amino acid sequence of SEQ ID NO: 1 (hereinafter, referred to as “R536K”), Taq DNA polymerase in which arginine was substituted with valine at amino acid residue 660 in the amino acid sequence of SEQ ID NO: 1 (hereinafter, referred to as “R660V”) and Taq DNA polymerase in which arginine was substituted with lysine at amino acid residue 536 and arginine was substituted with valine at amino acid residue 660 in the amino acid sequence of SEQ ID NO: 1 (hereinafter, referred to as “R536K/R660V”) were prepared as follows.

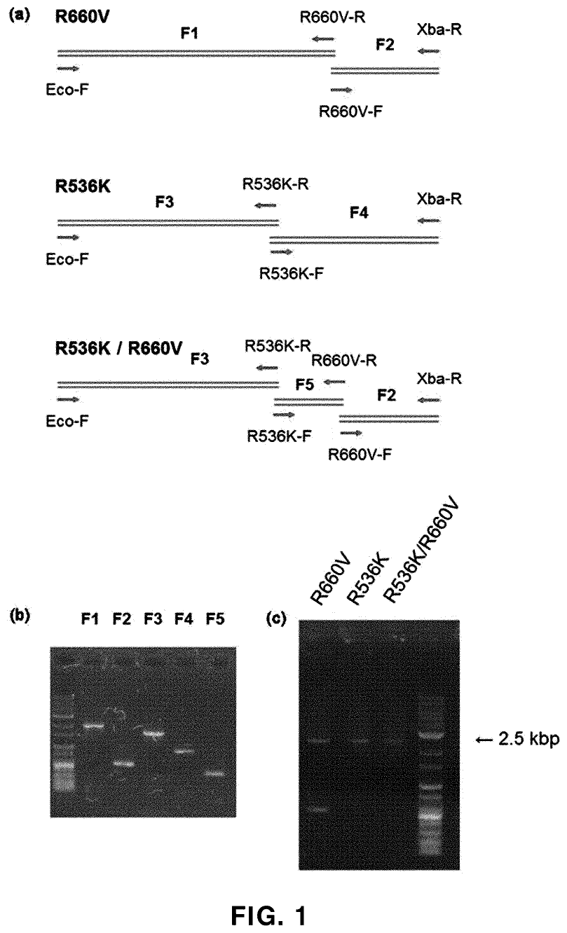

First, using mutation-specific primers described in Table 1, as shown in (a) in FIG. 1, Taq DNA polymerase fragments (F1 to F5) were amplified by PCR. Reaction conditions are as in Table 2.

TABLE 1

| Primer | Sequence (5′→3′) |
|---|---|
| Eco-F | GG GGTACC TCA TCA CCC CGG (SEQ ID NO: 17) |
| R536K-R | CTT GGT GAG CTC CTT GTA CTG CAG GAT |
| | (SEQ ID NO: 18) |
| R536K-F | ATC CTG CAG TAC AAG GAG CTC ACC AAG |
| | (SEQ ID NO: 19) |
| R660V-R | GAT GGT CTT GGC CGC CAC GCG CAT CAG GGG |
| | (SEQ ID NO: 20) |
| R660V-F | CCC CTG ATG CGC GTG GCG GCC AAG ACC ATC |
| | (SEQ ID NO: 21) |
| Xba-R | GC TCTAGA CTA TCA CTC CTT GGC GGA GAG CCA |
| | (SEQ ID NO: 22) |

TABLE 2

| | |
|---|---|
| 10× pfu buffer (SolGent) | 2.5 μl |
| dNTP (10 mM each) | 1 μl |
| F primer (10 pmol/μl) | 1 μl |
| R primer (10 pmol/μl) | 1 μl |
| Distilled water | 18 μl |
| pUC19-Tag (10 ng/μl) | 1 μl |
| Pfu polymerase | 0.5 μl |
| 30 cycles (Ta = 60° C.) | 25 μl |

PCR products were subjected to electrophoresis, thereby detecting a band for each fragment as shown in (b) in FIG. 1, indicating that a desired fragment was amplified.

1-2. Overlap PCR

Each amplified fragment obtained in 1-1 was used as a template, and full-length amplification thereof was performed using primers at both ends (Eco-F and Xba-R primers). Reaction conditions are as in Tables 3 and 4.

TABLE 3
R660V or R536K

| | |
|---|---|
| 10× pfu buffer (SolGent) | 5 μl |
| 5× enhancer (SolGent) | 10 μl |
| dNTP (10 mM each) | 1 μl |
| Eco-F primer (10 pmol/μl) | 2 μl |
| Xba-R primer (10 pmol/μl) | 2 μl |
| Distilled water | 27 μl |
| Fragment 1 (or fragment 3) | 1 μl |
| Fragment 2 (or fragment 4) | 1 μl |
| Pfu polymerase | 1 μl |
| 40 cycles (Ta = 62° C.) | 50 μl |

TABLE 4
R536K/R660V

| | |
|---|---|
| 10× pfu buffer (SolGent) | 5 μl |
| 5× enhancer (SolGent) | 10 μl |
| dNTP (10 mM each) | 1 μl |
| Eco-F primer (10 pmol/μl) | 2 μl |
| Xba-R primer (10 pmol/μl) | 2 μl |
| Distilled water | 26 μl |
| Fragment 2 | 1 μl |
| Fragment 3 | 1 μl |
| Fragment 5 | 1 μl |
| Pfu polymerase | 1 μl |
| 40 cycles (Ta = 62° C.) | 50 μl |

Consequently, as shown in (c) in FIG. 1, it, was confirmed that the Taq polymerases of “R536K,” “R660V” and “R536K/R660V” were amplified.

1-3. Ligation pUC19 was digested with restriction enzymes EcoRI/XbaI at 37° C. for 4 hours under conditions shown in Table 5 below, DNA was purified, and the purified DNA was treated with SAP at 37° C. for 1 hour under conditions shown in Table 6, thereby preparing a vector.

TABLE 5

| | |
|---|---|
| 10× CutSmart buffer (NEB) | 2.5 μl |
| pUC19 (500 ng/μl) | 21.5 μl |
| EcoRI-HF (NEB) | 0.5 μl |
| Xba I (NEB) | 0.5 μl |
| | 25 μl |

TABLE 6

| | |
|---|---|
| 10× SAP buffer (Roche) | 2 μl |
| Purified DNA | 17 μl |
| SAP (Roche) | 1 μl |
| | 20 μl |

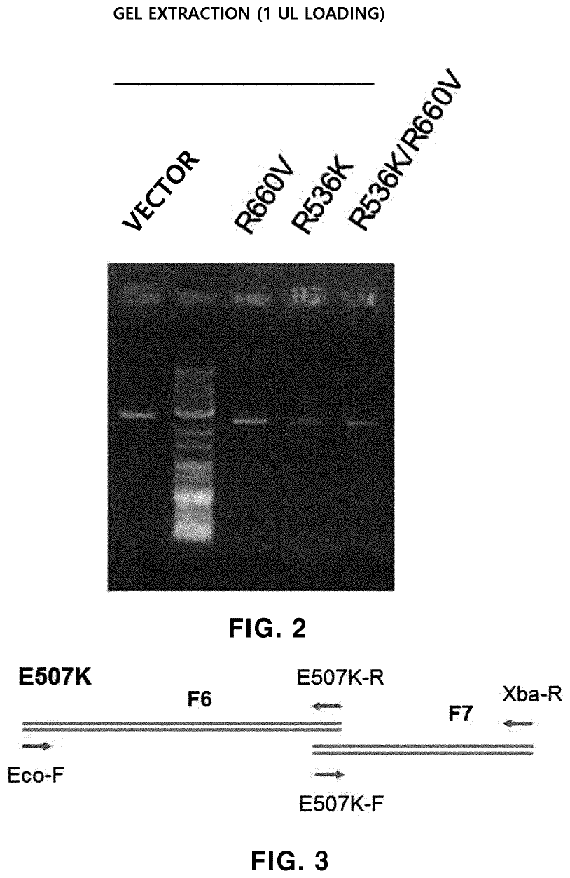

After the overlap PCR product was obtained in Example 1-2 and digested with restriction enzymes EcoRI/XbaI at 37° C. for 3 hours under conditions shown in Table 7, an insert was gel-extracted with the prepared vector (FIG. 2).

TABLE 7

| | |
|---|---|
| 10× CutSmart buffer (NEB) | 2 μl |
| Purified PCR product | 17 μl |
| EcoRI-HF (NEB) | 0.5 μl |
| XbaI (NEB) | 0.5 μl |
| | 20 μl |

After ligation was performed at room temperature for 2 hours under conditions shown in Table 8, E. coil DH5αwas transformed with the resulting vectors and then screened in a medium containing ampicillin. Plasmids prepared from the collected colonies were sequenced, thereby obtaining Taq DNA polymerase mutants (“R536K,” “R660V” and “R536K/R660V”) into which desired variation(s) is/are introduced.

TABLE 8

| | Vector only | Vector + Insert |
|---|---|---|
| 10× ligase buffer (SolGent) | 1 μl | 1 μl |
| Vector | 1 μl | 1 μl |
| Insert | — | 3 μl |
| Distilled water | 7 μl | 4 μl |
| T4 DNA ligase (SolGent) | 1 μl | 1 μl |
| | 10 μl | 10 μl |

### Example 2

Introduction of E507K Variation

2-1. Fragment PCR

The Taq polymerase activity of the “R536K,” “R660V” and “R536K/R660V” prepared in Example 1 was tested, thereby confirming that the activity was decreased (data not shown), the E507K variation (substitution of glutamic acid with lysine at amino acid residue 507 in the amino acid sequence of SEQ ID NO: 1) was additionally introduced into each of R536K, R660V and R536K/R660V, and the E507K variation was introduced into wild-type Taq DNA polymerase (WT) as a control. A method of preparing the E507K variation-introduced Taq DNA polymerase is the same as described in Example 1.

Taq DNA polymerase fragments (F6 to F7) shown in Table 3 were amplified by PCR using mutation-specific primers shown in Table 9. Reaction conditions are shown in Table 10.

TABLE 9

| Primer | Sequence (5′-3′) |
|---|---|
| Eco-F | GG GGTACC TCA TCA CCC CGG (SEQ ID NO: 17) |
| E507K-R | CTT GCC GGT CTT TTT CGT CTT GCC GAT |
| | (SEQ ID NO: 23) |
| E507K-F | ATC GGC AAG ACG AAA AAG ACC GGC AAG |
| | (SEQ ID NO: 24) |
| Xba-R | GC TCTAGA CTA TCA CTC CTT GGC GGA GAG CCA |
| | (SEQ ID NO: 22) |

TABLE 10

| | |
|---|---|
| 10× pfu buffer (SolGent) | 2.5 μl |
| dNTP (10 mM each) | 1 μl |
| F primer (10 pmol/μl) | 1 μl |
| R primer (10 pmol/μl) | 1 μl |
| Distilled water | 18 μl |
| Template plasmid (10 ng/μl) | 1 μl |
| Pfu polymerase | 0.5 μl |
| 30 cycles (Ta = 60° C.) | 25 μl |

*Template plasmids: pUC19-Taq (WT), pUC19-Taq (R536K), pUC19-Taq (R660V), and pUC19-Taq (R536K/R660V)

2-2. Overlap PCR

Full-length amplification was performed on each of the amplified fragments obtained in 2-1 as a template using primers (Eco-F and Xba-R primers) at both ends. Reaction conditions are shown in Table 11.

TABLE 11

| | |
|---|---|
| 10X pfu buffer (SolGent) | 5 μl |
| 5X enhancer (SolGent) | 10 μl |
| dNTP (10 mM each) | 1 μl |
| Eco-F primer (10 pmol/μl) | 2 μl |
| Xba-R primer (10 pmol/μl) | 2 μl |
| Distilled water | 27 μl |
| Fragment 6 | 1 μl |
| Fragment 7 | 1 μl |
| Pfu polymerase | 1 μl |
| 40 cycles (Ta = 62° C.) | 50 μl |

2-3. Ligation pUC19 was digested with restriction enzymes EcoRI/XbaI at 37° C. for 4 hours under conditions shown in Table 5 above. DNA was purified, the purified DNA was treated with SAP at 37° C. for 1 hour under conditions shown in Table 6, thereby preparing a vector.

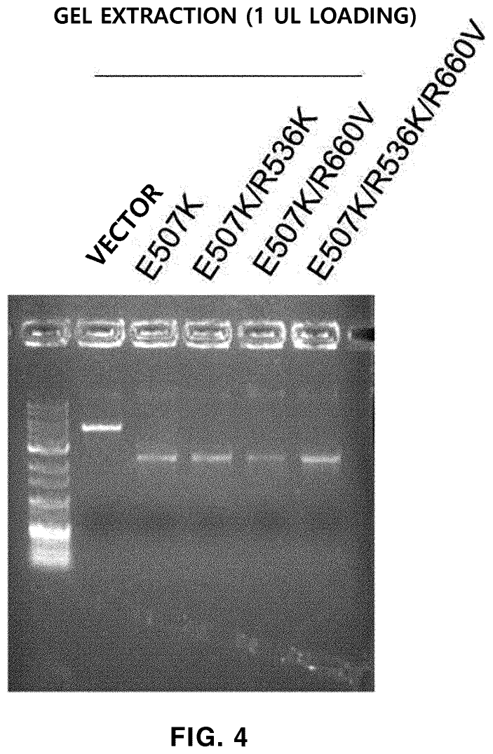

After the overlap PCR product was obtained in Example 2-2 and digested with restriction enzymes EcoRI/XbaI at 37° C. for 3 hours under conditions shown in Table 7, an insert was gel-extracted with the prepared vector (FIG. 4).

After ligation was performed at room temperature for 2 hours under conditions shown in Table 8, E. coli DH5α of DH10β has transformed with the resulting vectors and then screened in a medium containing ampicillin. Plasmids prepared from the collected colonies were sequenced, thereby obtaining E507K variation-introduced Taq DNA polymerase mutants (“E507K/R536K,” “E507K/R660V” and “E507K/R536K/R660V”)

### Example 3

Performance of qPCR Using DNA Polymerase of the Present Invention

The Tact polymerase having each of the “E507K/R536K,” “E507K/R660V” and “E507K/R536K/R660V” variations obtained in Example 2 was used to confirm whether an ability of extending a mismatched primer with respect to a template including a SNP was reduced. As a control, the “E507K” Taq polymerase having the E507K variation was used.

The templates including SNPs used herein are rs1408799, rs1015362 and rs4911414, and genotypes of the templates and sequence data of specific primers (IDT, USA) thereof are shown in Tables 12 and 13 below.

TABLE 12
Genotype of template

| | |
|---|---|
| rs1408799 | TT |
| rs1015362 | CC |
| rs4911414 | GG |

TABLE 13

| Primer Name | | Sequence (5′-3′) |
|---|---|---|
| rs1408799 | Forward | CCAGTGTTAGGTTATTTCTAACTTG |
| | | (SEQ ID NO: 25) |
| | Reverse_T | GCTCGGAGCACATGGTCAA |
| | | (SEQ ID NO: 26) |
| | Reverse_C | GCTCGGAGCACATGGTCAG |
| | | (SEQ ID NO: 27) |
| rs1015362 | Forward | TGAAGAGCAGGAAAGTTCTTCA |
| | | (SEQ ID NO: 28) |
| | Reverse_C | ACTGTGTGTCTGAAACAGTG |
| | | (SEQ ID NO: 29) |
| | Reverse_T | ACTGTGTGTCTGAAACAGTA |
| | | (SEQ ID NO: 30) |
| rs4911414 | Forward_G | GTAAGTCTTTGCTGAGAAATTCATTG |
| | | (SEQ ID NO: 31) |
| | Forward_T | GTAAGTCTTTGCTGAGAAATTCATTT |
| | | (SEQ ID NO: 32) |
| | Reverse | AGTATCCAGGGTTAATGTGAAAG |
| | | (SEQ ID NO: 33) |

Conditions for qPCR (Applied Biosystems 7500 Fast) are as shown in Table 14 below.

TABLE 14

| | |
|---|---|
| 95° C. 5 min | |
| 95° C. 20 sec | 50 cycles |
| 60° C. 30 sec | |
| 72° C. 30 sec | |
| 72° C. 3 min | |

Probes were dual-labeled as shown in Table 15 below.

TABLE 15

| Probe | | 5′ | 3′ |
|---|---|---|---|
| Name | Sequence (5′-3′) | fluorophore | quencher |
| 1408799- | AGATATTTGTAAGGTATTCTG | FAM | Black Hole |
| FAM | GCCT (SEQ ID NO: 34) | | Quencher 1 |
| 1015362- | TGCTGAACAAATAGTCCCGAC | HEX | Black Hole |
| HEX | CAG (SEQ ID NO: 35) | | Quencher 1 |
| 4911141- | TTTCTCTAGTTGCCTTTAAGA | Texas Red | Black Hole |
| Texas | TTT (SEQ ID NO: 36) | | Quencher 2 |
| Red | | | |

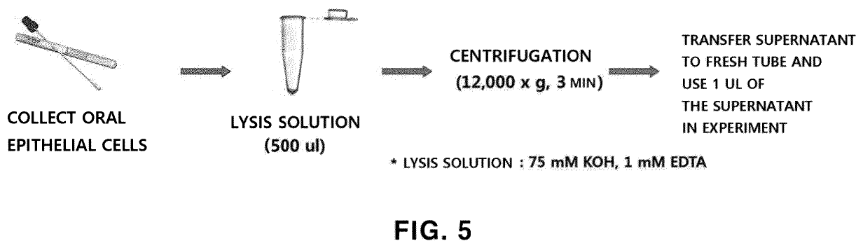

Oral epithelial cells were collected using a kit for collecting oral epithelial cells purchased from Noble Bio, lysed in 500 μl of a lysis solution, and then centrifuged at 12,000×g for 3 minutes. The supernatant was transferred to a fresh tube, and 1 μl per experiment was used (FIG. 5). Reaction conditions are shown in Table 16, and the composition of the reaction buffer is shown in Table 17.

TABLE 16

| | |
|---|---|
| 5X reaction buffer | 4 μl |
| 5M betaine | 2 μl |
| dNTP (10 mM each) | 0.5 μl |
| Forward primer (2 μM) | 1 μl |
| Reverse primer (2 μM) | 1 μl |
| Nuclease-free distilled water | 8 μl |
| Acquired template | 1 μl |
| Taq polymerase (2 U/μl) | 0.5 μl |
| Dual-labeled probe (4 μM) | 2 μl |
| 20 μl | |

TABLE 17

Reaction buffer (1X)
50 mM Tris-Cl (pH 8.8)
2.5 mM MgCl₂
50 mM KCl
5 mM (NH₄)₂SO₄
0.1% Tween 20
0.01% BSA

The other components of the reaction solution except a specific primer were prepared as shown in Table 13 in wo tubes, and each allele-specific primer was added thereto, thereby performing qPCR. Here, a difference in cycle (Ct) value at which combined fluorescent signals detected from the tubes reach the threshold fluorescence value calculated with AB 7500 software (v2.0.6) was analyzed. It is considered that, as the Ct value in the amplification by a mismatched primer is delayed, high gene variation specificity or allele specificity is exhibited. As a result of AS-qPCR for rs1408799, rs1015362 and rs4911414, as shown in FIGS. 6a-6d, 7 and 8, compared to the control E507K, when the Tap polymerase having E507K/R536K, E507K/R660V or E507K/R536K/R660V variations was used, it was confirmed that the amplification by a mismatched primer was delayed, and such an effect was most significantly exhibited in the E507K/R536K/R660V mutant.

It was confirmed. that the Tap DNA polymerase having the E507K/R536K, E507K/R660V or E507K/R536K/R660V variations according to the present invention, compared to that with E507K variation, has excellent mismatch extension selectivity. Therefore, it is expected that the three types of Taq DNA polymerases can be useful for medical diagnosis of a disease and recombinant DNA studies.

### Example 4

Introduction of 85871 Variation

4-1. Fragment PCR

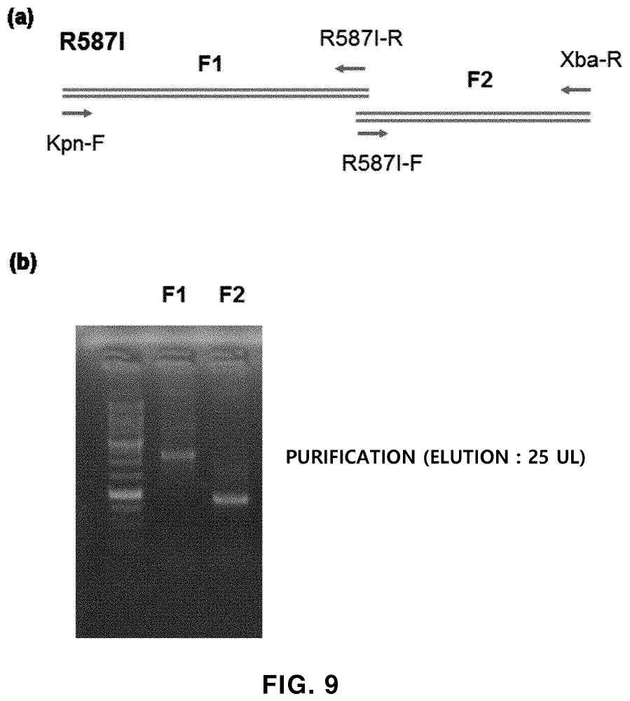

To additionally introduce a R5871 variation (substitution of arginine with isoleucine at amino acid residue 587 in the amino acid sequence of SEQ ID NO: 1) into the “E507K/R536K/R660V” variation-introduced Taq clone prepared in Example 2, as shown in (a) in FIG. 9, two fragments were amplified by PCR using primers shown in Table 18 below. Reaction conditions are shown in Table 19.

TABLE 18

| Primer | Sequence (5′-3′) |
|---|---|
| Kpn-F | TCC ACC CCG AGG GGT ACC TCA TCA CCC CGG CCT |
| | GGC (SEQ ID NO: 39) |
| R587I-R | CCC AAG CGG GGT GAT GAC GGG GAT GTT |
| | (SEQ ID NO: 40) |
| R587I-F | AAC ATC CCC GTC ATC ACC CCG CTT GGG |
| | (SEQ ID NO: 41) |
| Xba-R | CTG CAG GTC GAC TCT AGA CTA TCA CTC CTT GGC |
| | GGA G (SEQ ID NO: 42) |

TABLE 19

| | |
|---|---|
| 10X pfu buffer (SolGent) | 5 μl |
| dNTP (10 mM each) | 2 μl |
| F primer (10 pmol/μl) | 2 μl |
| R primer (10 pmol/μl) | 2 μl |
| Distilled water | 36 μl |
| Taq plasmid (E507K, R536K, R660V) | 2 μl |
| (10 ng/μl) | |
| Pfu polymerase | 1 μl |
| 35 cycles (Ta = 60° C.) | 50 μl |

The PCR product was confirmed by electrophoresis, and thus, as shown in (b) FIG. 9, a band for each fragment was continued, indicating that a desired fragment was amplified.

4-7. In-Fusion Cloning

A Taq plasmid vector (E507K/R536K/R660V) was digested with restriction enzymes KpnI/XbaI at 37° C.; for 4 hours under conditions shown in Table 20 and then purified (elution: 25 μl), thereby preparing an open linear vector. Afterward, an in-fusion cloning reaction was performed under conditions shown in Table 21 at 37° C. for 15 minutes to transform E. coli DH5α or DH10β, and then the transformed cell was screened in an ampicillin-containing medium. Plasmids prepared from the collected colonies were sequenced, thereby obtaining a R5871 variation-introduced Taq DNA polymerase mutant (“E507K/R536K/R587I/R660V”).

TABLE 20

| | |
|---|---|
| 10X CutSmart Vector (NEB) | 2.5 μl |
| Taq plasmid (E507K/R536K/R660V) | 21.5 μl |
| (200 ng/μl) | |
| Kpn I-HF (NEB) | 0.5 μl |
| Xba I (NEB) | 0.5 μl |
| 25 μl | |

TABLE 21

| | |
|---|---|
| 5X EZ-fusio mix (Enzynomics) | 2 μl |
| Vector cleaved with Kpn I, Xba I | 1 μl |
| (50 ng/μl) | |
| F1 fragment (83 ng/μl) | 1 μl |
| F2 fragment (50 ng/μl) | 1 μl |
| Distilled water | 5 μl |
| 10 μl | |

### Example 5

Performance of qPCR Using “E507K/R536K/R587I/R660V” Taq Polymerase

5-1. Discrimination of Q61H variations in KRAS Gene

The Taq polymerase having the “E507K/R536K/R587I/R660V” variations obtained in Example 4 was used to confirm whether an ability of extending mismatched primers with respect to templates with Q61H SNPs in the KRAS gene was reduced. As a control, the Taq polymerase having “E507K/R536K/R660V” variations was used.

The template including a SNP was gDNA (104 copies, 33 ng/rxn) obtained from a HepG2 liver cancer cell line, and obtained by a typical DNA extraction method. It was confirmed that an entire detected target site corresponds to the NCBI reference sequence (NG_007524.1), and used as a wild-type (WT).

The sequence data of specific primers for the template is shown in Table 22 below.

TABLE 22

| Primer | | | Tm |
|---|---|---|---|
| Name | | Sequence (5′-3′) | (° C.) |
| KRAS | Forward_Q | GAT ATT CTC GAC ACA GCA | 64.2 |
| Q61H | (24 mer) | GGT CAA (SEQ ID NO: 43) | |
| | Forward_H | GAT ATT CTC GAC ACA GCA | 64.4 |
| | (24 mer) | GGT CAC (SEQ ID NO: 44) | |
| | Reverse | ACA AAG AAA GCC CTC CCC | 64.2 |
| | | AG (SEQ ID NO: 45) | |

Conditions for qPCR (Applied Biosystems 7500 Fast) are the same as shown in Table 14 in Example 3. Probes are labeled as shown Table 23 below.

TABLE 23

| Probe | | Tm |
|---|---|---|
| Name | Sequence (5′-3′) | (° C.) |
| Q61H FAM | TGC AAT GAG GGA CCA GTA | 67.6 |
| | CAT GAG G (SEQ ID NO: 46) | |

Reaction conditions are the same as shown in Table 16 in Example 3, and the composition of the reaction buffer is the same as in Table 24 below.

TABLE 24

Reaction buffer (1X)
50 mM Tris-Cl (pH 8.8)
2.5 mM MgCl₂
60 mM KCl
2.5 mM (NH₄)₂SO₄
25 mM TMAC
0.1% Tween 20
0.01% BSA

The other components of the reaction solution except a specific primer were prepared as shown in Table 22 in two tubes, and each allele-specific primer was added thereto, thereby performing qPCR. Here, a difference in cycle (Ct) value at which combined fluorescent signals detected from the tubes reach the threshold fluorescence value calculated with AB 7500 software (v2.0.6) was analyzed. It is considered that, as the Ct value in the amplification by a mismatched primer is delayed, high gene variation specificity or allele specificity is exhibited. As a result of AS-qPCR, as shown in FIGS. 10a and 10b, compared to the control E507K/R536K/R660V, the Taq polymerase having E507K/R536K/R587I/R660V variations was increased in ΔCt up to 5, indicating that the amplification by a mismatched primer was delayed.

The inventors further performed the above-described experiment once again using a primer shown in Table 25, which was manufactured by shortening the 24-mer primer of Table 22 to 18 mer. Except for using the composition of the reaction buffer in Table 26 below, all conditions are the same as those in the experiment using the 24-mer primer.

TABLE 25

| Primer | | | Tm |
|---|---|---|---|
| Name | | Sequence (5′-3′) | (° C.) |
| KRAS | Forward_Q | CTC GAC ACA GCA GGT CAA | 61.4 |
| Q61H | (18 mer) | (SEQ ID NO: 47) | |
| | Forward_H | CTC GAC ACA GCA GGT CAC | 61.8 |
| | (18 mer) | (SEQ ID NO: 48) | |
| | Reverse | ACA AAG AAA GCC CTC CCC | 64.2 |
| | | AG (SEQ ID NO: 49) | |

TABLE 26

Reaction buffer (1X)
50 mM Tri-Cl (pH 8.8)
2.5 mM MgCl₂
15 mM (NH₄)₂SO₄
0.1% Tween 20
0.01% BSA

Consequently, as shown in FIGS. 10c and 10d, compared to the control E507K/R536K/R660V, the Taq polymerase having E507K/R536K/R587I/R660V variations can confirm that the amplification by a mismatched primer is delayed. Particularly, the ΔCt of the R587I-introduced polymerase was more remarkably increased.

5-2. Discrimination of G13D Variations in KRAS Gene

The Taq polymerase having the “E507K/R536K/R587I/R660V” variations obtained in Example 4 was used to confirm whether an ability of extending mismatched primers with respect to templates with G13D SNPs in the KRAS gene was reduced. As a control Taq polymerase having “E507K/R536K/R660V” variations was used.

The template including an SNP was gDNA (104 copies, 33 ng/rxn) obtained from a HepG2 liver cancer cell line, and obtained by a typical DNA extraction method. It, was confirmed that an entire detected target site corresponds to the NCBI reference sequence (NG_007524.1), and used as a wild-type (WT).

The sequence data of specific primers for the template is shown in Table 27 below.

TABLE 27

| Primer | | | Tm |
|---|---|---|---|
| Name | | Sequence (5′-3′) | (° C.) |
| KRAS | Forward | ATA AGG CCT GCT GAA AAT GAC | 61 |
| G13D | | (SEQ ID NO: 50) | |
| | Reverse_G | GGC ACT CTT GCC TAC GC | 62.4 |
| | (17 mer) | (SEQ ID NO: 51) | |
| | Reverse_D | GGC ACT CTT GCC TAC GT | 61.2 |
| | (17 mer) | (SEQ ID NO: 52) | |

Conditions for qPCR (Applied Biosystems 7500 Fast) are the same as shown in Table 14 in Example 3. Probes are labeled as shown in Table 28 below.

TABLE 28

| Probe | | Tm |
|---|---|---|
| Name | Sequence (5′-3′) | (° C.) |
| G1213_R | AGC TCC AAC TAC CAC AAG TTT ATA | 66.2 |
| FAM | TTC AGT (SEQ ID NO: 53) | |

Reaction conditions are the same as shown in Table 16 in Example 3, and the composition of the reaction buffer is the same as in Table 24 in Example 5-1. The other components of the reaction solution except a specific primer were prepared as shown in Table 27 in two tubes, and each allele-specific primer was added thereto, thereby performing qPCR. Here, a difference in cycle (Ct) value at which combined fluorescent signals detected from the tubes reach the threshold fluorescence value calculated with AB 7500 software (v2.0.6) was analyzed. It is considered that, as the Ct value in the amplification by a mismatched primer is delayed, high gene variation specificity or allele specificity is exhibited.

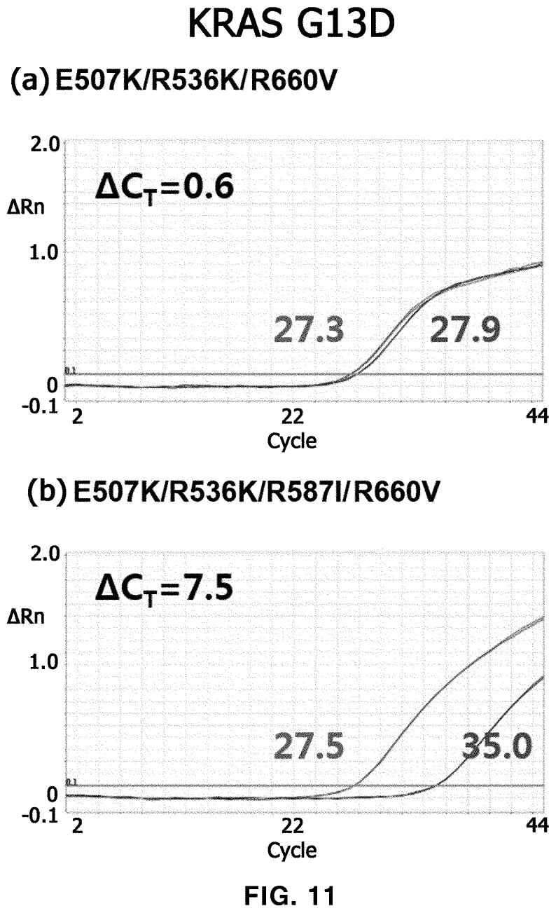

As a Result of AS-qPCR, as Shown in FIG. 11, Compared to the Control

E507K/R536K/R660V, the Taq polymerase having E507K/R536K/R587I/R660V variations confirmed that the amplification by a mismatched primer was delayed.

5-3. Discrimination of G12S Variations in KRAS Gene

The Taq polymerase having the “E507K/R536K/R587I/R660V” variations obtained in Example 4 was used to confirm whether an ability of extending mismatched primers with respect to templates having G13S SNPs in the KRAS gene was reduced. As a control, the Taq polymerase having “E507K/R536K/R660V” variations was used.

The template having an SNP was gDNA (104 copies, 33 ng/ rxn) obtained from a HepG2 liver cancer cell line, and obtained by a typical DNA extraction method. It was confirmed that an entire detected target site corresponds to the NCBI reference sequence (NG_007524.1.), and used as a wild-type (WT).

The sequence data of specific primers for the template is shown in Table 29 below.

TABLE 29

| Primer | | | Tm |
|---|---|---|---|
| Name | | Sequence (5′-3′) | (° C.) |
| KRAS | Forward_G | TAA ACT TGT GGT AGT TGG | 62.6 |
| G125 | (23 mer) | AGC TG (SEQ ID NO: 54) | |
| | Forward_S | TAA ACT TGT GGT AGT TGG | 61.6 |
| | (23 mer) | AGC TA (SEQ ID NO: 55) | |
| | Reverse | CAT ATT CGT CCA CAA AAT | 63 |
| | | GAT TCT GAA T (SEQ ID | |
| | | NO: 56) | |

Conditions for qPCR (Applied Biosystems 7500 Fast) are the same as shown in Table 14 in Example 3. Probes are labeled as shown in Table 30 below.

TABLE 30

| | | Tm |
|---|---|---|
| Probe Name | Sequence (5′-3′) | (° C.) |
| G1213_F FAM | AGC TGT ATC GTC AAG GCA | 68.2 |
| | CTC TTG C (SEQ ID NO: 57) | |

Reaction conditions are the same as shown in Table 16 in Example 3, and the composition of the reaction buffer is the same as in Table 24 in Example 5-1. The other components of the reaction solution except a specific primer were prepared as shown in Table 29 in two tubes, and each allele-specific primer was added thereto, thereby performing qPCR. Here, a difference in cycle (Ct) value at which combined fluorescent signals detected from the tubes reach the threshold fluorescence value calculated with AB 7500 software (v2.0.6) was analyzed. It is considered that, as the Ct value in the amplification by a mismatched primer is delayed, high gene variation specificity or allele specificity is exhibited.

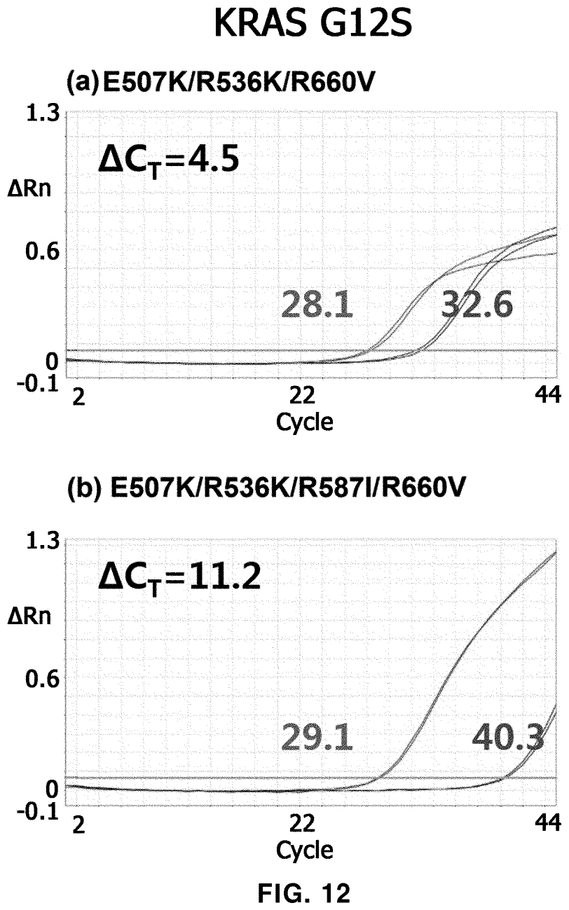

As a result of AS-qPCR, as shown in FIG. 12, compared to the control E507K/R536K/R660V, the Taq polymerase having E507K/R536K/R587I/R660V variations confirmed that the amplification by a mismatched primer was delayed.

5-4. Discrimination of L858R Variations in EGER Gene 5 The Taq polymerase having the “E5071(1R536K/R5871/R660 V” variations obtained in Example 4 was used to confirm whether an ability of extending mismatched primers with respect to templates with L858R SNPs in EGFR gene was reduced. As a control, Taq polymerase having “E507K/R536K/R660V” variations was used.

The template including a SNP was gDNA (104 copies, 33 ng/rxn) obtained from a HepG2 liver cancer cell line, and obtained by a typical DNA extraction method. It was continued that an entire detected target site corresponds to the NCBI reference sequence (NG_007726.3), and used as a wild-type (WT).

The sequence data of specific primers for the template is shown in Table 31 below.

TABLE 31

| Primer | | | |
|---|---|---|---|
| Name | | Sequence (5′-3′) | Tm (° C.) |
| EGFR | Forward | ACC TGG CAG CCA GGA | 67.8 |
| L858R | | ACG TA (SEQ ID NO: 58) | |
| | Reverse_L | GCA CCC AGC AGT TTG | 68.2 |
| | | GCC A (SEQ ID NO: 59) | |
| | Reverse_R | GCA CCC AGC AGT TTG | 67.7 |
| | | GCC C (SEQ ID NO: 60) | |

Conditions for qPCR (Applied Biosystems 7500 Fast) are the same as shown in Table 14 in Example 3. Probes are labeled as shown in Table 32 below.

TABLE 32

| Probe Name | Sequence (5′-3′) | Tm (° C.) |
|---|---|---|
| L858R FAM_R | CAG CAT GTC AAG ATC ACA GAT | 67.8 |
| | TTT GGG C (SEQ ID NO: 61) | |

Reaction conditions are the same as shown in Table 16 in Example 3, and the composition of the reaction buffer is the same as in Table 24 in Example 5-1. The other components of the reaction solution except a specific primer were prepared as shown in Table 31 in two tubes, and each allele-specific primer was added thereto, thereby performing qPCR. Here, a difference in cycle (Ct) value at which combined fluorescent signals detected from the tubes reach the threshold fluorescence value calculated with AB 7500 software (v2.0.6) was analyzed. It is considered that, as the Ct value in the amplification by a mismatched primer is delayed, high gene variation specificity or allele specificity is exhibited.

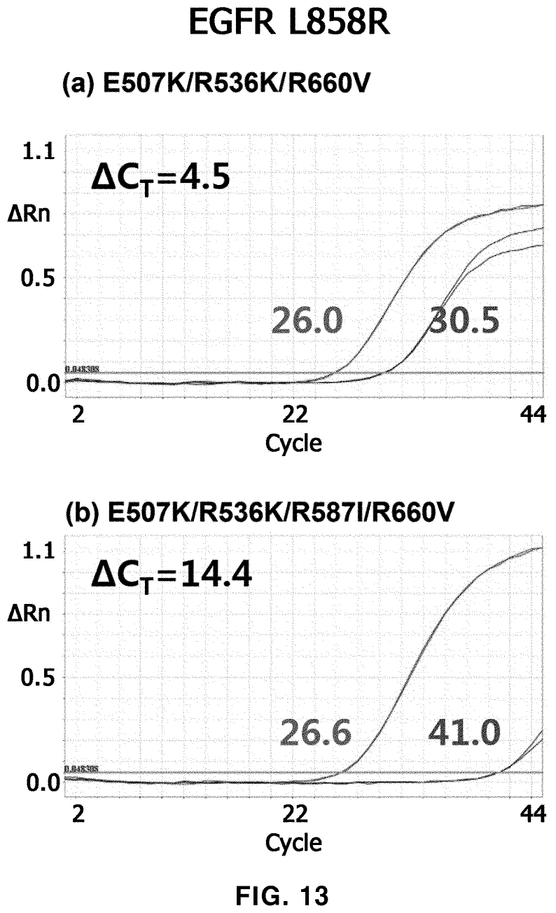

As a result of AS-qPCR, as shown in FIG. 13, compared to the control E507K/R536K/R660V, the Taq polymerase having E507K/R536K/R587I/R660V variations confirmed that the amplification by a mismatched primer was delayed.

As described above, it was confirmed that some of the Taq DNA polymerases having E507K/R536K/R587R660V variations according to the present invention, compared to the Taq polymerase having E507K/R536K/R660V variations, have excellent mismatch extension selectivity. Therefore, the Taq DNA polymerases having E507K/R536K/R587I/R660V variations according to the present invention are also expected to be usefully applied to the medical diagnosis of a disease and. recombinant DNA studies.

### Example 6

Optimization of KCl Concentration in Reaction Buffer

In this example, to find a high cation concentration in a state in which the amplification by mismatching is delayed as much as possible, and the amplification efficiency by matching is not reduced, an optimal KCl concentration was confirmed by adjusting a KCl concentration in a PCR buffer.

The Taq polymerases having “E507K/R536K,” “E507K/R660V” and “E507K/R536K/R660V” variations, respectively, obtained in Example 2 were used to compare a KCl concentration threshold with the Taq polymerase having the E507K variation.

As a template having an SNP, rs1408799 was used, the genotype of the template was TT, and as a primer, an rs1408799 primer shown in Table 2 was used. qPCR (Applied Biosystems 7500 Fast) was performed under the conditions shown in Table 14, a dual-labeled probe is 1408799-FAM shown in Table 15, the reaction conditions are shown in Table 33, and the composition of the reaction buffer is shown in Table 34.

TABLE 33

| | |
|---|---|
| 5X Reaction buffer | 4 μl |
| dNTP (10 mM each) | 0.5 μl |
| Forward primer (2 μM) | 1 μl |
| Reverse primer (2 μM) | 1 μl |
| Nuclease-free distilled water | 10 μl |
| Acquired template (TT) | 1 μl |
| Taq polymerase (2 U/μl) | 0.5 μl |
| Dual-labeled probe (4 μM) | 2 μl |
| 20 μl | |

TABLE 34

Reaction buffer (1X)
50 mM Tris-Cl (pH 8.8)
2.5 mM MgCl₂
x mM KCl
2.5 mM (NH₄)₂SO₄
0.1% Tween 20
0.01% BSA

Consequently, as shown in FIGS. 14a to 14d, it was confirmed that the E507K/R536K/R660V Taq polymerase has the lowest KCl concentration threshold, and the E507K/R536K and E507K/R660V have lower KCl concentration thresholds than E507K. Based on the result, to determine the optimal KCl concentration, an additional experiment was performed using the E507K/R536K/R660V Taq 10 polymerase. A primer was the rs1408799-T-specific primer shown in Table 13, qPCR (Applied Biosystems 7500 Fast) was performed for 35 cycles under the conditions shown in Table 14, and reaction conditions are shown in Table 35.

TABLE 35

| | |
|---|---|
| 5X Reaction buffer | 4 μl |
| dNTP (10 mM each) | 0.5 μl |
| Forward primer (2 μM) | 1 μl |
| Reverse primer (2 μM) | 1 μl |
| Nuclease-free distilled water | 12 μl |
| Acquired template (TT) | 1 μl |
| E507K/R536K/R660V (2 U/μl) | 0.5 μl |
| 20 μl | |

The composition of a reaction buffer for the control is shown in Table 36, and the composition of a reaction buffer for the experimental group is shown in Table 34.

The (NH₄)7SO₄ concentration was constantly fixed at 2.5 mM, and the KCl concentration varied.

TABLE 36

Control buffer (1X)
50 mM Tris-Cl (pH 8.8)
1M betaine
2.5 mM MgCl₂
50 mM KCl
2.5 mM (NH₄)₂SO₄
0.1% Tween 20
0.01% BSA

Amplification was performed under the above-mentioned conditions, and the PCR product was identified by electrophoresis, thereby confirming that, as shown in FIG. 15, an optimal KCl concentration in a state in which the amplification by mismatching is delayed as much as possible, and the amplification efficiency by matching is not reduced is 75 mM.

### Example 7

Optimization of (NH₄)₂SO₄ Concentration in Reaction Buffer

In this example, based on the result of Example 4, the optimal (NH₄)₂SO₄ concentration was confirmed. by constantly fixing a KCl concentration in a reaction buffer at 75 mM and variously changing a (NH₄)₂SO₄ concentration. As a primer, the rs1408799-T-specific primer shown in Table 13 was used, qPCR (Applied Biosystems 7500 Fast) was performed for 35 cycles under the conditions shown in Table 14, reaction conditions are shown in Table 35, and the composition of a reaction buffer for the control is shown in Table 36.

Consequently, as shown in FIG. 16, it was confirmed that an optimal (NH₄)₂SO₄ concentration is 5 mM.

Based on the result, an amplification delay effect caused by mismatching was further confirmed by constantly fixing the KCl concentration in the reaction buffer at 75 mM, and setting the (NH₄)₂SO₄ concentration to approximately 5 mM (each of 2.5 mM, 5 mM and 10 mM).

As a primer, the rs1.408799 primer shown in Table 13 was used, a dual-labeled probe is 1408799-FAM shown in Table 15, and reaction conditions are shown in Table 37 below.

TABLE 37

| | |
|---|---|
| 5X Reaction buffer | 4 μl |
| dNTP (10 mM each) | 0.5 μl |
| Forward primer (2 μM) | 1 μl |
| Reverse primer (2 μM) | 1 μl |
| Nuclease-free distilled water | 10 μl |
| Acquired template (TT) | 1 μl |
| E507K/R536K/R660V (2 U/μl) | 0.5 μl |
| Dual-labeled probe (4 μM) | 2 μl |
| 20 μl | |

Consequently, as shown in FIG. 17, when the (NH₄)₂SO₄ concentration was 10 mM, the Ct value difference was the largest, but Ct was a little delayed and a peak was tilted in the amplification caused by matching, and the optimal (NH₄)₂SO₄ concentration was determined to be 5 mM. By combining the results of Examples 6 and 7, it was confirmed that the optimal composition of a reaction buffer contains 50 mM Tris.Cl, 2.5mM MgCl₂, 75 mM KCl, 5 mM (NH₄)₂SO₄, 0.1% Tween 20 and 0.01% BSA.

### Example 8

Addition of TMAC to Reaction Buffer and Optimization of TMAC Concentration

In this example, the optimal concentration was confirmed by adding TMAC to a reaction buffer. Based on the results of Examples 6 and 7, the optimal TMAC concentration was determined by constantly fixing a KCl concentration at 75 mM and a (NH₄)₂SO₄ concentration at 5 mM, and variously changing a TMAC concentration.

A E507K/R536K or E507K/R536K/R660V Taq polymerase was used, and as a template having an SNP, rs1408799 was used. The genotype of the template was TT, and as a primer, the rs1408799 primer shown in Table 13 was used. qPCR (Applied Biosystems 7500 Fast) was performed under the conditions shown in Table 14, a dual-labeled probe is 1408799-FAM shown in Table 15, and reaction conditions are shown in Table 37.

Consequently, as shown in FIGS. 18a and 18b, it was confirmed that, for the E507K/R536K Taq polymerase, the optimal TMAC concentration is 60 mM, and for the E507K/R536K/R660V Taq polymerase, the optimal TMAC concentration is 25 mM. When the TMAC concentration is very high, amplification efficiency was reduced.

### Example 9

Optimization of KCl, (NH₄)₂SO₄ and TMAC Concentrations in Reaction Buffer

In this Example, based on the result shown in Example 8, the optimal KCl, (NH₄)₂SO₄ and TMAC concentrations in a reaction buffer were confirmed using the E507K/R536K/R660V Taq polymerase.

Specifically, the TMAC concentration was constantly fixed at 25 mM, the (NH₄)₂SO₄ concentration was constantly fixed at 2.5 nM, and then the KCl concentration was changed to 20, 40, 60 or 80 mM. An experiment was performed on two SNPs of rs1015362 and rs4911414, and the genotype of the template is shown in Table 12, primers were the rs1015362 and rs4911414 primers shown in Table 13, qPCR (Applied Biosystems 7500 Fast) was performed under the conditions shown in Table 14, a dual-labeled probe is 1408799-TAM shown in Table 15, and reaction conditions are shown in Table 37.

Consequently, as shown in FIGS. 19a and 19b the optimal KCl concentration for two SNPs was 60 mM, and it can be observed that when the KCl 80 mM, amplification efficiency was reduced.

From the above-described results, it was confirmed that the optimal KCl concentration in the reaction buffer was 60 mM, the optimal (NH₄)₂SO₄ concentration was 2.5 mM, and the optimal TMAC concentration was 25 mM, and in further detail, for the E507K/R536K polymerase, 75 mM KCl 5 mM (NH₄)₂SO₄ and 60 mM TMAC were most effectively used, and for the E507K/R536K/R660V polymerase, 60 mM KCl, 2.5 mM (NH₄)₂SO₄ and 25 mM TMAC were most effectively used.

### INDUSTRIAL APPLICABILITY

Since the DNA polymerase with increased gene variation specificity according to the present invention has a higher mismatch-to-match extension selectivity than conventional Taq polymerase, reliable gene variation-specific amplification is possible without any substrate modify. The present invention provides an optimal PCR buffer composition that allows the proper function of a DNA polymerase with increased gene variation specificity to be effectively exhibited, and reliable gene variation-specific amplification is possible by considerably increasing the activity of the DNA polymerase using the DNA polymerase with increased gene variation specificity. Moreover, a kit including a buffer composition and/or the DNA polymerase with increased gene variation specificity according to the present invention can effectively detect a gene variation or SNP, and thus can be usefully applied to the medical diagnosis of a disease and recombinant DNA studies.

What is claimed is:

1. A PCR buffer composition for increasing the activity of a DNA polymerase with increased gene variation-specific amplification efficiency, the composition comprising 25 to 100 mM HCl and 1 to 15 mM (NH₄)₂SO₄, wherein the final pH is 8.0 to 9.0.

2. The PCR buffer composition of claim 1, wherein the KCl concentration is 60 to 90 mM.

3. The PCR buffer composition of claim 1, wherein the (NH₄)₂SO₄ concentration is 2.5 to 8 mM.

4. The PCR buffer composition of claim 1, wherein the KCl concentration is 70 to 80 mM, and the (NH₄)₂SO₄ concentration is 4 to 6 mM.

5. The PCR buffer composition of claim 1, further comprising 5 to 80 mM tetra methyl ammonium chloride (TMAC).

6. The PCR buffer composition of claim 5, wherein the KCl concentration is 40 to 90 mM.

7. The PCR buffer composition of claim 5, wherein the (NH₄)₂SO₄ concentration is 1 to 7 mM.

8. The PCR buffer composition of claim 5, wherein the TMAC concentration is 15 to 70 mM, the KCl concentration is 50 to 80 mM, and the (NH₄)₂SO₄ concentration is 1.5 to 6 mM.

9. The PCR buffer composition of claim 1, further comprising Tris.Cl and MgCl2.

10. A PCR kit for detecting a gene variation or SNP, comprising the PCR buffer composition of claim 1.

11. The PCR kit of claim 10, further comprising the following DNA polymerase, which is DNA polymerase comprising a Taq polymerase amino acid sequence of SEQ ID NO: 1, comprising: (a) a substitution at amino acid residue 507 in the amino acid sequence of SEQ ID NO: 1; and (b) (i) a substitution at amino acid residue 536 in the amino acid sequence of SEQ ID NO: 1, (ii) a substitution at amino acid residue 660 in the amino acid sequence of SEQ ID NO: 1, (iii) substitutions at amino acid residues 536 and 660 in the amino acid sequence of SEQ ID NO: 1, or (iv) substitutions at amino acid residues 536, 587 and 660 in the amino acid sequence of SEQ ID NO: 1.

12. The PCR kit of claim 11, wherein the substitution at the amino acid residue 507 is a substitution of glutamic acid (E) with lysine (K), the substitution at the amino acid residue 536 is a substitution of arginine (R) with lysine (K), the substitution at the amino acid residue 587 is a substitution of arginine (R) with isoleucine (I), and the substitution at the amino acid residue 660 is a substitution of arginine (R) with valine (V).

13. The PCR kit of claim 10, further comprising:
a) a nucleoside triphosphate;
b) a quantification reagent binding to double-stranded DNA;
c) a polymerase blocking antibody;
d) one or more control values or control sequences; and
e) one or more templates.

14. A method of in vitro detecting one or more gene variations or SNPs in one or more templates by using the PCR kit of claim 10.

15. The method of claim 14, wherein the PCR kit further comprises the following DNA polymerase, which is a DNA polymerase comprising a Taq polymerase amino acid sequence of SEQ ID NO: 1, comprising: (a) a substitution at amino acid residue 507 in the amino acid sequence of SEQ ID NO: 1; and (b) (i) a substitution at amino acid residue 536 in the amino acid sequence of SEQ ID NO: 1, (ii) a substitution at amino acid residue 660 in the amino acid sequence of SEQ ID NO: 1, (iii) substitutions at amino acid residues 536 and 660 in the amino acid sequence of SEQ ID NO: 1, or (iv) substitutions at amino acid residues 536, 587 and 660 in the amino acid sequence of SEQ 111) NO: 1.

16. The method of claim 15, wherein the substitution at the amino acid residue 507 is a substitution of glutamic acid (E) with lysine (K), the substitution at the amino acid residue 536 is a substitution of arginine (R) with lysine (K), the substitution at the amino acid residue 587 is a substitution of arginine (R) with isoleucine (I), and the substitution at the amino acid residue 660 is a substitution of arginine (R) with valine (V).
